(19) United States
(12) Patent Application Publication
(10) Pub. No.: US 2017/0023519 A1
(43) Pub. Date: Jan. 26, 2017

(54) WEARABLE DEVICES INCORPORATING ION SELECTIVE FIELD EFFECT TRANSISTORS

(71) Applicant: Fitbit, Inc., San Francisco, CA (US)

(72) Inventors: Aaron Alexander Rowe, Toluca Lake, CA (US); Thomas Samuel Elliot, San Francisco, CA (US); Javier L. Prieto, Oakland, CA (US)

(21) Appl. No.: 15/282,976

(22) Filed: Sep. 30, 2016

Related U.S. Application Data
(63) Continuation of application No. 15/199,720, filed on Jun. 30, 2016.

(51) Int. Cl. G01N 27/414; H01L 29/45; H01L 23/02; A61B 5/00; A41C 3/00; A61B 5/01; A61B 5/145; A61B 5/1477; G01N 27/416; H01L 23/34

(52) U.S. Cl. CPC G01N 27/414; G01N 27/4167; H01L 29/45; H01L 23/02; H01L 23/34; A41C 3/0057; A61B 5/01; A61B 5/14517; A61B 5/14539; A61B 5/14546; A61B 5/1477; A61B 5/0022; A61B 5/443; A61B 5/6804; A61B 5/681; A61B 5/742; A61B 5/7475; A61B 2560/0242

(57) ABSTRACT

Techniques for measuring ion related metrics at a user's skin surface are disclosed. In one aspect, a method for operating a wearable device may involve determining, based on output of one or more ion selective field effect transistor sensors, various physiological conditions such as a state of hydration, a state of skin health, or the cleanliness of the wearable device or an associated garment.

### INCORPORATION BY REFERENCE TO RELATED APPLICATION

Any and all priority claims identified in the Application Data Sheet, or any correction thereto, are hereby incorporated by reference under 37 CFR 1.57. This application is a continuation of U.S. application Ser. No. 15/199,720, filed on Jun. 30, 2016. The aforementioned application is incorporated by reference herein in its entirety, and is hereby expressly made a part of this specification.

### TECHNICAL FIELD

This disclosure relates to the field of wearable devices, and particularly to the measurement of physiological or other properties therewith.

### BACKGROUND

Consumer interest in personal health has led to a variety of personal health monitoring devices being offered on the market. Such devices, until recently, tended to be complicated to use and were typically designed for use with one activity, for example, bicycle trip computers.

Advances in sensors, electronics, and power source miniaturization have allowed the size of personal health monitoring devices, also referred to herein as “biometric tracking,” “biometric monitoring,” or simply “wearable” devices, to be offered in extremely small sizes that were previously impractical. The number of applications for these devices is increasing as the processing power and component miniaturization for wearable devices improves.

Many wearable chemical sensors have been invented, but none are fit for commercialization as a consumer product. Wearable sensors that monitor body chemistry can be divided into several categories, based upon the materials that are used in these sensing devices, and the way that the sensing element makes contact with the body. Each of these categories of sensor has drawbacks. Continuous glucose monitors, worn by some patients with Type 1 or Type 2 Diabetes, are the largest class of on-the-market wearable chemical sensors. A major drawback of these sensors is that the sensing element is in the form of a needle that must be inserted into the skin, causing pain and irritation. The pain and irritation caused by inserting the sensing element into the skin makes these sensors unattractive for use by athletes, generally healthy people, and other individuals who do not have diabetes. For the most part, these sensors are meant for days of implantation in the skin.

Screen printed electrodes can be pressed against the skin. The surface area of these sensors is generally large and the materials used to fabricate these sensors are often fragile. Most screen printed sensors employ an enzyme that acts as a transducer, generating an electrochemically detectable byproduct while digesting the analyte. Enzymes are a class of proteins, and proteins are intrinsically fragile and perishable. Furthermore, proteins are capable of triggering inflammation when they come into contact with the skin. Temporary tattoo type sensors, which sit above the skin, are generally fabricated through screen printing and thus are intrinsically thin and fragile. The application of a polymer, enzyme, and reagent mix directly onto the surface of the skin is likely to cause irritation. Subdermal tattoo type sensors, embedded in the skin, are a likely source of irritation. Furthermore, the user may not tolerate the pain associated with the tattooing process.

Transcutaneous blood gas sensors make use of a liquid or gel filled drum which is heated and pressed against the skin. Gasses from the skin diffuse into the liquid, causing a change in signals at a pH sensor and oxygen sensor. Handling of these sensors is labor intensive as they have replaceable membranes and they must be periodically refilled with gel. Electrochemical biosensors with a needle-like sensor are inserted deep into the skin, e.g., as in the continuous glucose sensors used by diabetes patients. Furthermore, the insertion of a needle coated in a potentially irritating set of substances is not acceptable to many users. Microneedle patches have been inserted into the skin and used for biosensing, but despite the small diameter of each individual microneedle, irritation remains a problem.

Electrochemical sensor watches including the OV™ watch and GlucoWatch™ place an electrochemical sensing instrument on the wrist. The GlucoWatch™ was withdrawn from the market because it caused irritation. The OV™ watch is also no longer on the market. Both of these sensors contained fragile disposable modules. A device containing an ISFET, for the measurement of vaginal pH has been developed, but the commercialization status of this device is unclear. Optical sensors that measure light reflected from the skin or scattered from the skin can employ Raman spectroscopy and have a demonstrated ability to measure the levels of analytes beneath the surface of the skin, but the power demands of these systems are very high, necessitating bulky power supplies. Furthermore, exotic and costly optical components are used in these systems, and the band of infrared that is employed is not well transmitted by darker skin types.

### SUMMARY

The systems, methods and devices of this disclosure each have several innovative aspects, no single one of which is solely responsible for the desirable attributes disclosed herein.

The devices and methods of the embodiments utilize as a sensing element an ion-selective field effect transistor (ISFET), or an array containing a set of ISFETs. There is a linear relationship between the temperature of an ion sensor and its output. For this reason, ion sensors are preferably accompanied by temperature sensors. Often this temperature sensor is integrated into the same chip as the ISFET device itself Variants of ISFETs can also be employed in devices and methods of certain embodiments. These variants include a chemical field-effect transistor (CHEMFET), an ENFET (i.e., a CHEMFET specialized for detection of specific biomolecules using enzymes, wherein an enzyme is attached to the gate area of an ISFET, giving it the ability to recognize and measure the levels of a specific chemical), and a MEMFET (i.e., a membrane-equipped ISFET). In each of these cases, an accessory is added to an ion-selective field effect transistor, giving it the ability to recognize a specific chemical species. The devices and methods of the embodiments facilitate monitoring over the course of months, and the devices can endure many wash and rinse cycles and the harsh environment of the body.

In a first aspect, a wearable device is provide, comprising: an ion selective field effect transistor; and a reference electrode, wherein the ion selective field effect transistor and the reference electrode are configured to be in direct contact with a user's skin.

In an embodiment of the first aspect, the reference electrode is selected from the group consisting of an Ag/AgCl electrode, an Ag/AgCl plastic composite electrode, an Ag/AgCl gel electrode, an Ag/AgCl electrode coated with a permeable membrane, a polypyrrole electrode, a conductive polymer material doped with one or more mediators, a conductive polymer material, and a poly(3,4-ethylenedioxythiophene) electrode.

In an embodiment of the first aspect, the permeable membrane is selected from the group consisting of polyvinyl butyral, polyhydroxyethylmethacrylate, nafion, and combinations thereof.

In an embodiment of the first aspect, the reference electrode is an Ag/AgCl electrode coated with a permeable membrane and saturated with chloride ions.

In an embodiment of the first aspect, the one or more mediators comprise a mediator selected from the group consisting of prussian blue, ferrocene, ferrocene derivatives, and combinations thereof.

In an embodiment of the first aspect, the reference electrode is a carbon paste electrode mixed with a mediator, e.g., the mediator can be ferrocene or prussian blue, and, e.g., the permeable membrane is polyvinyl butyral, nafion, or polyhydroxyethylmethacrylate.

In an embodiment of the first aspect, the reference electrode is a noble metal reference electrode and/or a pseudo reference electrode, e.g., the noble metal can be gold or platinum, and, e.g., the noble metal reference electrode and/or the pseudo reference electrode is paired with a reference field effect transistor.

In an embodiment of the first aspect, the device further comprises a temperature sensor, wherein the temperature sensor is integrated with the ion selective field effect transistor, and wherein the temperature sensor is configured to be in direct contact with the user's skin when the wearable device is in use.

In an embodiment of the first aspect, the ion selective field effect transistor is incorporated into a housing, wherein a portion of the ion selective field effect transistor is situated on a protrusion of the housing configured to enhance skin contact with the portion.

In an embodiment of the first aspect, at least one of the ion selective field effect transistor and the reference electrode are configured for integration into a wristband, a sports bra, or a waistband.

In an embodiment of the first aspect, the ion selective field effect transistor is configured to monitor a first characteristic of a fluid at a surface of the user's skin, wherein the monitoring is continuous and long term, and wherein the characteristic is selected from the group consisting of pH, electrolytic conductivity, Na⁺ concentration, and K⁺ concentration.

In an embodiment of the first aspect, the device further comprises at least one additional ion selective field effect transistor, wherein the additional ion selective field effect transistor is configured to monitor a second characteristic of fluid at a surface of the user's skin, wherein the second characteristic is different from the first characteristic.

In a second aspect, a wearable device is provided, comprising: an ion selective field effect transistor configured to be in direct contact with a user's skin; a reference electrode configured to be in direct contact with the user's skin; a user interface; at least one processor; and a memory storing computer-executable instructions for controlling the at least one processor to: determine, based on output of the ion selective field effect transistor, at least one of pH and an ion concentration; provide, via the user interface, information indicative of the pH or the ion concentration in a fluid at a surface of the user's skin.

In an embodiment of the second aspect, the processor is configured to sample the output of the ion selective field effect transistor at a faster rate when the user is physically active than when the user is sedentary.

In an embodiment of the second aspect, the processor is configured to sample the output at the faster rate after the user has been physically active for at least a predetermined period.

In an embodiment of the second aspect, the predetermined period is ten or more minutes.

In an embodiment of the second aspect, the reference electrode is selected from the group consisting of an Ag/AgCl electrode, an Ag/AgCl plastic composite electrode, an Ag/AgCl gel electrode, an Ag/AgCl electrode coated with a permeable membrane, a polypyrrole electrode, a poly(3,4-ethylenedioxythiophene) electrode, a doped or undoped conductive polymer material, a conductive polymer material doped with one or more mediators, and a conductive polymer doped with ferrocene and/or ferrocene derivatives.

In an embodiment of the second aspect, the ion selective field effect transistor is incorporated into a housing, wherein a portion of the ion selective field effect transistor is situated on a protrusion of the housing configured to enhance skin contact with the portion.

In a third aspect, a wearable device is provided, comprising: an ion selective field effect transistor configured to be in direct contact with a user's skin; a reference electrode configured to be in direct contact with the user's skin; and a processor, wherein the processor is configured to sample the output of the ion selective field effect transistor at a faster rate when the user is physically active than when the user is sedentary.

In an embodiment of the third aspect, the processor is configured to sample the output at the faster rate after the user has been physically active for at least a predetermined period.

In an embodiment of the third aspect, the predetermined period is ten or more minutes.

In an embodiment of the third aspect, the reference electrode is selected from the group consisting of an Ag/AgCl electrode, an Ag/AgCl plastic composite electrode, an Ag/AgCl gel electrode, an Ag/AgCl electrode coated with a permeable membrane, a polypyrrole electrode, a poly(3,4-ethylenedioxythiophene) electrode, a doped or undoped conductive polymer material, a conductive polymer material doped with one or more mediators, and a conductive polymer doped with ferrocene and/or ferrocene derivatives.

In an embodiment of the third aspect, at least one of the ion selective field effect transistor and the reference electrode are configured for integration into a wristband, a sports bra, or a waistband.

In a fourth aspect, a wearable device is provided, comprising: an ion selective field effect transistor; and a reference electrode, wherein the ion selective field effect transistor is incorporated into a housing, wherein a portion of the ion selective field effect transistor is situated on a protrusion of the housing configured to enhance skin contact with the portion.

In an embodiment of the fourth aspect, the reference electrode is incorporated into the housing.

In an embodiment of the fourth aspect, the reference electrode is in electrical communication with the ion selective field effect transistor via a wired connection.

In a fifth aspect, a wearable device for monitoring cleanliness of the wearable device or a garment associated with the wearable device is provided, comprising: an ion selective field effect transistor; and a reference electrode, wherein the ion selective field effect transistor and the reference electrode are configured to be in direct contact with a user's skin.

In an embodiment of the fifth aspect, the reference electrode is selected from the group consisting of an Ag/AgCl electrode, an Ag/AgCl plastic composite electrode, an Ag/AgCl gel electrode, an Ag/AgCl electrode coated with a permeable membrane, a polypyrrole electrode, and a poly(3,4-ethylenedioxythiophene) electrode.

In an embodiment of the fifth aspect, the reference electrode comprises a conductive polymer material, wherein the conductive polymer is undoped or doped.

In an embodiment of the fifth aspect, the conductive polymer material is doped with one or more mediators.

In an embodiment of the fifth aspect, the one or more mediators comprise ferrocene and/or ferrocene derivatives.

In an embodiment of the fifth aspect, the device further comprises a temperature sensor, wherein the temperature sensor is integrated with the ion selective field effect transistor, and wherein the temperature sensor is configured to be in direct contact with the user's skin when the wearable device is in use.

In an embodiment of the fifth aspect, the ion selective field effect transistor is incorporated into a housing, wherein a portion of the ion selective field effect transistor is situated on a protrusion of the housing configured to enhance skin contact with the portion.

In an embodiment of the fifth aspect, at least one of the ion selective field effect transistor and the reference electrode are configured for integration into a wristband, a sports bra, or a waistband.

In an embodiment of the fifth aspect, the ion selective field effect transistor is configured to monitor a first characteristic of a fluid at a surface of the user's skin, wherein the monitoring is continuous and long term, and wherein the characteristic is selected from the group consisting of pH, electrolytic conductivity, Na⁺ concentration, and K⁺ concentration.

In an embodiment of the fifth aspect, the device further comprises at least one additional ion selective field effect transistor, wherein the additional ion selective field effect transistor is configured to monitor a second characteristic of fluid at a surface of the user's skin, wherein the second characteristic is different from the first characteristic.

In a sixth aspect, method is provided for operating a wearable device for monitoring cleanliness of the wearable device or a garment associated with the wearable device, the wearable device comprising an ion selective field effect transistor, a reference electrode, and a user interface, the method comprising: measuring, based on output of the ion selective field effect transistor, a characteristic of a fluid present on a user's skin; determining, based on the measured characteristic of the fluid, an amount of residue buildup on the wearable device or the garment exceeding a threshold amount, wherein the residue buildup comprises one or more components selected from the group consisting of soap residue buildup, grease residue buildup, skin cream residue buildup, and sunblock residue buildup; and providing, via the user interface, information indicative of a cleanliness of the wearable device or the garment.

In an embodiment of the sixth aspect, the characteristic is pH, and wherein the threshold amount is exceeded when a pH greater than 7 is measured by the ion selective field effect transistor.

In an embodiment of the sixth aspect, the threshold amount is exceeded when a pH greater than 7.5 is measured by the ion selective field effect transistor.

In an embodiment of the sixth aspect, the characteristic is pH, and wherein the threshold amount is user configured or user specified.

In an embodiment of the sixth aspect, the characteristic is pH, and wherein the threshold amount is set by an algorithm and/or logic on the wearable device or a server in communication with the wearable device.

In an embodiment of the sixth aspect, the server is a cloud software service.

In an embodiment of the sixth aspect, the characteristic is ion concentration, and wherein the threshold amount is exceeded when an ion concentration greater than X (e.g., 1.1) times a physiological maximum ion concentration in eccrine sweat is measured by the ion selective field effect transistor.

In a seventh aspect, a wearable device for monitoring cleanliness of the wearable device or a garment associated with the wearable device is provided, comprising: an ion selective field effect transistor; a reference electrode; and a user interface; at least one processor; and a memory storing computer-executable instructions for controlling the at least one processor to: measure, based on output of the ion selective field effect transistor, a characteristic of a fluid present on a user's skin; determine, based on the measured characteristic of the fluid, an amount of residue buildup on the wearable device or the garment exceeding a threshold amount, wherein the residue buildup comprises one or more components selected from the group consisting of soap residue buildup, grease residue buildup, skin cream residue buildup, and sunblock residue buildup; and provide, via the user interface, information indicative of a cleanliness of the wearable device or the garment.

In an embodiment of the seventh aspect, the processor is configured to sample the output of the ion selective field effect transistor at a faster rate when the user is physically active than when the user is sedentary.

In an embodiment of the seventh aspect, the processor is configured to sample the output at the faster rate after the user has been physically active for at least a predetermined period.

In an embodiment of the seventh aspect, the predetermined period is ten or more minutes.

In an embodiment of the seventh aspect, the device further comprises a temperature sensor, wherein the temperature sensor is integrated with the ion selective field effect transistor, and wherein the temperature sensor is configured to be in direct contact with the user's skin when the wearable device is in use.

In an embodiment of the seventh aspect, the reference electrode is incorporated into a housing.

In an embodiment of the seventh aspect, the ion selective field effect transistor is incorporated into a housing, wherein a portion of the ion selective field effect transistor is situated on a protrusion of the housing configured to enhance skin contact with the portion.

In an embodiment of the seventh aspect, at least one of the ion selective field effect transistor and the reference electrode are configured for integration into a wristband, a sports bra, or a waistband.

In an embodiment of the seventh aspect, the device further comprises at least one additional ion selective field effect transistor, wherein the additional ion selective field effect transistor is configured to monitor a second characteristic of fluid at a surface of the user's skin, wherein the second characteristic is different from the first characteristic.

In an eighth aspect, a wearable device for monitoring cleanliness of the wearable device or a garment associated with the wearable device is provided, comprising: an ion selective field effect transistor; a reference electrode; and a user interface; at least one processor; and a memory storing computer-executable instructions for controlling the at least one processor to: determine, based on output of the ion selective field effect transistor, at least one of pH and an ion concentration; determine, based on the measured characteristic of the fluid, an amount of soap residue buildup on the wearable device or the garment exceeding a threshold amount; and provide, via the user interface, information indicative of a cleanliness of the wearable device or the garment.

In an embodiment of the eighth aspect, the processor is configured to sample the output of the ion selective field effect transistor at a faster rate when the user is physically active than when the user is sedentary.

In an embodiment of the eighth aspect, the processor is configured to sample the output at the faster rate after the user has been physically active for at least a predetermined period.

In an embodiment of the eighth aspect, the predetermined period is ten or more minutes.

In a ninth aspect, a wearable device for monitoring hydration of a user is provided, comprising: an ion selective field effect transistor; and a reference electrode, wherein the ion selective field effect transistor and the reference electrode are configured to be in direct contact with a user's skin.

In an embodiment of the ninth aspect, the reference electrode is selected from the group consisting of an Ag/AgCl electrode, an Ag/AgCl plastic composite electrode, an Ag/AgCl gel electrode, an Ag/AgCl electrode coated with a permeable membrane, a polypyrrole electrode, and a poly(3,4-ethylenedioxythiophene) electrode.

In an embodiment of the ninth aspect, the reference electrode comprises a conductive polymer material, wherein the conductive polymer is undoped or doped.

In an embodiment of the ninth aspect, the conductive polymer material is doped with one or more mediators.

In an embodiment of the ninth aspect, the one or more mediators comprise ferrocene and/or ferrocene derivatives.

In an embodiment of the ninth aspect, the device further comprises a temperature sensor, wherein the temperature sensor is integrated with the ion selective field effect transistor, and wherein the temperature sensor is configured to be in direct contact with the user's skin when the wearable device is in use.

In an embodiment of the ninth aspect, the ion selective field effect transistor is incorporated into a housing, wherein a portion of the ion selective field effect transistor is situated on a protrusion of the housing configured to enhance skin contact with the portion.

In an embodiment of the ninth aspect, at least one of the ion selective field effect transistor and the reference electrode are configured for integration into a wristband, a sports bra, or a waistband.

In an embodiment of the ninth aspect, the ion selective field effect transistor is configured to monitor a first characteristic of a fluid at a surface of the user's skin, wherein the monitoring is continuous and long term, and wherein the characteristic is selected from the group consisting of pH, electrolytic conductivity, Na⁺ concentration, and K⁺ concentration.

In an embodiment of the ninth aspect, the device further comprises at least one additional ion selective field effect transistor, wherein the additional ion selective field effect transistor is configured to monitor a second characteristic of fluid at a surface of the user's skin, wherein the second characteristic is different from the first characteristic.

In a tenth aspect, a method is provided for operating a wearable device for monitoring hydration of a user, the wearable device comprising an ion selective field effect transistor, a reference electrode, and a user interface, the method comprising: measuring, based on output of the ion selective field effect transistor, an ion concentration of a fluid at a surface of a user's skin; determining, based on the measured ion concentration of the fluid, a state of hydration of the user; and providing, via the user interface, information indicative of the user's state of hydration.

In an embodiment of the tenth aspect, measuring the ion concentration of a fluid at a surface of the user comprises sampling the output of the ion selective field effect transistor at a faster rate when the user is physically active than when the user is sedentary.

In an embodiment of the tenth aspect, the output is sampled at the faster rate after the user has been physically active for at least a predetermined period.

In an embodiment of the tenth aspect, the predetermined period is ten or more minutes.

In an embodiment of the tenth aspect, information is provided indicative of a state of dehydration if a first derivative of a sodium concentration of the fluid (e.g., sodium concentration of sweat at the user's skin) with respect to time exceeds a threshold set by the user or set by an algorithm and/or logic on the wearable device or a server in communication with the wearable device.

In an embodiment of the tenth aspect, the server is a cloud software service.

In an eleventh aspect, a wearable device for monitoring a state of hydration of a user is provided, comprising: an ion selective field effect transistor; a reference electrode; and a user interface; at least one processor; and a memory storing computer-executable instructions for controlling the at least one processor to: measure, based on output of the ion selective field effect transistor, an ion concentration of a fluid at a surface of a user's skin; determine, based on the measured ion concentration, a state of hydration of the user; and provide, via the user interface, information indicative of the user's state of hydration.

In an embodiment of the eleventh aspect, the processor is configured to sample the output of the ion selective field effect transistor at a faster rate when the user is physically active than when the user is sedentary.

In an embodiment of the eleventh aspect, the processor is configured to sample the output at the faster rate after the user has been physically active for at least a predetermined period.

In an embodiment of the eleventh aspect, the predetermined period is ten or more minutes.

In an embodiment of the eleventh aspect, the device further comprises a temperature sensor, wherein the temperature sensor is integrated with the ion selective field effect transistor, and wherein the temperature sensor is configured to be in direct contact with the user's skin when the wearable device is in use.

In an embodiment of the eleventh aspect, the reference electrode is incorporated into a housing.

In an embodiment of the eleventh aspect, the ion selective field effect transistor is incorporated into a housing, wherein a portion of the ion selective field effect transistor is situated on a protrusion of the housing configured to enhance skin contact with the portion.

In an embodiment of the eleventh aspect, at least one of the ion selective field effect transistor and the reference electrode are configured for integration into a wristband, a sports bra, or a waistband.

In an embodiment of the eleventh aspect, the device further comprises at least one additional ion selective field effect transistor, wherein the additional ion selective field effect transistor is configured to monitor a second characteristic of fluid at a surface of the user's skin, wherein the second characteristic is different from the first characteristic.

In a twelfth aspect, a wearable device for monitoring a state of hydration of a user is provided, comprising: an ion selective field effect transistor; a reference electrode; and a user interface; at least one processor; and a memory storing computer-executable instructions for controlling the at least one processor to: measure, based on output of the ion selective field effect transistor, an ion concentration of a fluid at a surface of a user's skin; activate, by the processor, at least one dehydration detection subroutine, wherein the dehydration detection subroutine is activated by a period of physical activity and/or exercise (e.g., as determined by sensors of the wearable device); determine, based on the measured ion concentration, a state of hydration of the user; and provide, via the user interface, information indicative of the user's state of hydration.

In an embodiment of the twelfth aspect, the processor is configured to sample the output of the ion selective field effect transistor at a faster rate when the user is physically active than when the user is sedentary.

In an embodiment of the twelfth aspect, the processor is configured to sample the output at the faster rate after the user has been physically active for at least a predetermined period.

In an embodiment of the twelfth aspect, the predetermined period is ten or more minutes.

In a thirteenth aspect, a wearable device for monitoring skin health is provided, comprising: an ion selective field effect transistor; and a reference electrode, wherein the ion selective field effect transistor and the reference electrode are configured to be in direct contact with a user's skin.

In an embodiment of the thirteenth aspect, the reference electrode is selected from the group consisting of an Ag/AgCl electrode, an Ag/AgCl plastic composite electrode, an Ag/AgCl gel electrode, an Ag/AgCl electrode coated with a permeable membrane, a polypyrrole electrode, and a poly(3,4-ethylenedioxythiophene) electrode.

In an embodiment of the thirteenth aspect, the reference electrode comprises a conductive polymer material, wherein the conductive polymer is undoped or doped.

In an embodiment of the thirteenth aspect, the conductive polymer material is doped with one or more mediators.

In an embodiment of the thirteenth aspect, the one or more mediators comprise ferrocene and/or ferrocene derivatives.

In an embodiment of the thirteenth aspect, the device further comprises a temperature sensor, wherein the temperature sensor is integrated with the ion selective field effect transistor, and wherein the temperature sensor is configured to be in direct contact with the user's skin when the wearable device is in use.

In an embodiment of the thirteenth aspect, the ion selective field effect transistor is incorporated into a housing, wherein a portion of the ion selective field effect transistor is situated on a protrusion of the housing configured to enhance skin contact with the portion.

In an embodiment of the thirteenth aspect, at least one of the ion selective field effect transistor and the reference electrode are configured for integration into a wristband, a sports bra, or a waistband.

In an embodiment of the thirteenth aspect, the ion selective field effect transistor is configured to monitor a first characteristic of a fluid at a surface of the user's skin, wherein the monitoring is continuous and long term, and wherein the characteristic is selected from the group consisting of pH, electrolytic conductivity, Na+ concentration, and K+ concentration.

In an embodiment of the thirteenth aspect, the device further comprises at least one additional ion selective field effect transistor, wherein the additional ion selective field effect transistor is configured to monitor a second characteristic of fluid at a surface of the user's skin, wherein the second characteristic is different from the first characteristic.

In a fourteenth aspect, a method is provided for operating a wearable device for monitoring skin health, the wearable device comprising an ion selective field effect transistor, a reference electrode, and a user interface, the method comprising: measuring, based on output of the ion selective field effect transistor, a characteristic of a fluid at a surface of a user's skin, wherein the characteristic is selected from the group consisting of a pH and an ion concentration; determining, based on the measured ion concentration of the fluid, an indicator of health of the user's skin; and providing, via the user interface, information indicative of the health of the user's skin.

In an embodiment of the fourteenth aspect, a pH of 6 or greater is an indicator of skin irritation or poor skin health.

In an embodiment of the fourteenth aspect, a specific pH threshold value is an indicator of skin irritation or poor skin health, and wherein the specific pH threshold value is user configured or user specified.

In an embodiment of the fourteenth aspect, a specific pH threshold value is an indicator of skin irritation or poor skin health, and wherein the specific pH threshold value is set by an algorithm and/or logic on the wearable device or a server in communication with the wearable device.

In an embodiment of the fourteenth aspect, the server is a cloud software service.

In an embodiment of the fourteenth aspect, the method further comprises sensing a UV absorption value for the user's skin, wherein the determining comprises determining, based on output of the ion selective field effect transistor and the UV sensor, an indicator of health of the user's skin.

In an embodiment of the fourteenth aspect, the user interface comprises at least one of a display, a light-emitting circuit, a sound-producing circuit, and a haptic drive circuit.

In an embodiment of the fourteenth aspect, the wearable device further comprises a transceiver configured to communicate with a client device.

In an embodiment of the fourteenth aspect, the client device comprises one of a personal computer, a mobile phone, and a tablet computing device.

In a fifteenth aspect, a wearable device for monitoring skin health is provided, comprising: an ion selective field effect transistor; a reference electrode; a user interface; at least one processor; and a memory storing computer-executable instructions for controlling the at least one processor to: measure, based on output of the ion selective field effect transistor, a characteristic of a fluid at a surface of a user's skin, wherein the characteristic is selected from the group consisting of a pH and an ion concentration;

determine, based on the measured characteristic of the fluid, an indicator of health of the user's skin; and provide, via the user interface, information indicative of the health of the user's skin.

In an embodiment of the fifteenth aspect, the processor is configured to sample the output of the ion selective field effect transistor at a faster rate when the user is physically active than when the user is sedentary.

In an embodiment of the fifteenth aspect, the processor is configured to sample the output at the faster rate after the user has been physically active for at least a predetermined period.

In an embodiment of the fifteenth aspect, the predetermined period is ten or more minutes.

In an embodiment of the fifteenth aspect, the device further comprises a temperature sensor, wherein the temperature sensor is integrated with the ion selective field effect transistor, and wherein the temperature sensor is configured to be in direct contact with the user's skin when the wearable device is in use.

In an embodiment of the fifteenth aspect, the reference electrode is incorporated into a housing.

In an embodiment of the fifteenth aspect, the ion selective field effect transistor is incorporated into a housing, wherein a portion of the ion selective field effect transistor is situated on a protrusion of the housing configured to enhance skin contact with the portion.

In an embodiment of the fifteenth aspect, at least one of the ion selective field effect transistor and the reference electrode are configured for integration into a wristband, a sports bra, or a waistband.

In an embodiment of the fifteenth aspect, the device further comprise at least one additional ion selective field effect transistor, wherein the additional ion selective field effect transistor is configured to monitor a second characteristic of fluid at a surface of the user's skin, wherein the second characteristic is different from the first characteristic.

In a sixteenth aspect, a wearable device for monitoring skin health is provided, comprising: an ion selective field effect transistor; a reference electrode; and a user interface; at least one processor; one or more biometric sensors configured to determine a physiological metric of the user, wherein the measured physiological metric is used by the processor to improve an accuracy of the information provided via the user interface.

In an embodiment of the sixteenth aspect, the processor is configured to sample the output of the ion selective field effect transistor at a faster rate when the user is physically active than when the user is sedentary.

Any of the features of an embodiment of the first through sixteenth aspects is applicable to all aspects and embodiments identified herein. Moreover, any of the features of an embodiment of the first through sixteenth aspects is independently combinable, partly or wholly with other embodiments described herein in any way, e.g., one, two, or three or more embodiments may be combinable in whole or in part. Further, any of the features of an embodiment of the first through sixteenth aspects may be made optional to other aspects or embodiments. Any aspect or embodiment of a method can be performed by a system or apparatus of another aspect or embodiment, and any aspect or embodiment of a system or apparatus can be configured to perform a method for another aspect or embodiment.

### BRIEF DESCRIPTION OF THE DRAWINGS

FIG. 1 is a rendering of a side view and a perspective view of a wearable device 10 that integrates an ISFET sensor on a protrusion 11 in accordance with aspects of this disclosure. The data from the wearable device 10 can be displayed on a user interface 12.

FIG. 2 is a block diagram showing how signals from a sensor module including an ISFET, MEMS accelerometer, a heart rate sensor 21 are employed in the processing and contextualization of embedded signals 22 from an ISFET sensor, and cloud based signal contextualization 23, in accordance with aspects of this disclosure.

FIG. 3 is a schematic of an electrical circuit for a p-ISFET of a wearable device in accordance with aspects of this disclosure.

FIG. 4. provides a schematic depicting a device 40 including integration of an ISFET sensor 41 and temperature sensor 42 on a sensor chip 49 with a readout circuit 43, ADC 45 and processor 46, and reference electrode 44, in accordance with aspects of this disclosure.

FIG. 5 is schematic of a sensor 50 comprising an ISFET and a reference electrode 54 of a wearable device in accordance with aspects of this disclosure. The ISFET includes a source 56, a drain 55, a response membrane (gate) 51, a gap 52 of 100 micrometers, and a silicon substrate 53.

FIG. 6. provides a schematic depicting integration of an ISFET sensor 61 and temperature sensor 62 on a sensor chip 69 with a readout circuit 63, ADC 65 and processor 66, and reference electrode 64, wherein analog sensors 68 including an optical heart rate sensor and a capacitance sensor are connected to the ADC via multiple connections 67, in accordance with aspects of this disclosure.

FIG. 7 is a block diagram illustrating certain components of an example wearable device in accordance with aspects of this disclosure.

FIG. 8 is a block diagram illustrating example biometric sensors which may be in communication with a processor of a wearable device in accordance with aspects of this disclosure.

FIG. 9 is an example of a wrist-worn device in accordance with aspects of this disclosure.

FIG. 10 is graph depicting pH data obtained from a human subject wearing a prototype ISFET device on his wrist while walking a treadmill.

FIG. 11 is a block diagram showing how signals from the ISFET of the wearable device are used to monitor cleanliness of the wearable device or a garment associated with the wearable device.

FIG. 12 is a block diagram showing how signals from the ISFET of the wearable device are used to determine a user's state of hydration.

FIG. 13 is a block diagram showing how signals from the ISFET of the wearable device are used to determine a user's skin health.

### DETAILED DESCRIPTION

One of the applications of wearable devices may be the monitoring of a physiological metric of a user of a wearable device via at least one biometric sensor. Such physiological metrics can include characteristics of a fluid at a surface of a user's skin, e.g., pH, ion concentration (e.g., Na⁺, K⁺, Cl⁺), electrolytic conductivity, or the like. Various algorithms or techniques can be applied to processing such physiological metric data, so as to provide information regarding a physiological condition of the user or a condition of an associated article. For example, information regarding a state of hydration of the user, or a user's degree of skin irritation or skin health may be determined. Similarly, the cleanliness of the wearable device and/or an associated garment can be determined by metrics indicative of the presence of residue buildup from soap, grease, skin cream, and/or sunblock on the device and/or an associated garment. Data from the biometric sensor can be obtained at a predetermined sampling rate that can be optionally be adjusted based upon whether the user is physically active or in a sedentary state, and alerts can be provided for certain conditions. The threshold for alerts may be predefined or user selected based on a user's unique physical characteristics.

Although the techniques of this disclosure may be described in connection with the determination of physiological metrics by a wearable device integrated with an ion selective field effect transistor (ISFET), this disclosure is not limited to the use of an ISFET. Other sensing technologies may be used in place of, or in addition to, an ISFET, including those sensors configured for measuring ions as are known in the art. ISFET technology offers the advantage in that they can advantageously be employed for measuring physiological metrics, e.g., pH, pCO₂, nitrogen ion, and potassium ion levels in sweat, other bodily fluids (e.g., blood, saliva, or interstitial fluid), or in other fluids, whether biological and/or environmental in nature, at a surface of the body, e.g., the skin. ISFET technology is durable and suitable for long term use, e.g., they can withstand cleaning using soapy water and a cloth or scrub brush.

In related aspects, one or more ISFETs, each tailored to measurement of a particular metric, optionally with additional sensors or electrodes, may be provided on a support or board of suitable size for integration with a wearable device as described in detail herein, e.g., of a size and shape suitable for a wristband. The side of the support with the one or more ISFETs is configured to be placed in contact with the skin, this side being referred to as the front side. The side of the support with the one or more ISFETs can optionally include a temperature sensor. Each ISFET is in electrical communication with a reference electrode. The reference electrode can be incorporated into the board so as to be in contact with the skin, or can be located elsewhere, e.g., on another portion of the wearable device in contact with the skin (e.g., the wristband) or in an associated garment in contact with the skin, e.g., a tee-shirt, a jacket, a sports bra, briefs, a waistband, a wristband, a headband, a sock, or the like. In certain embodiments, the reference electrode is located on a replaceable watch band, and this band can interface with an electrical contact on the body of the device, referred to herein as the ‘pebble’. In some embodiments, the entire watch band may be made from a reference electrode material such as a fabric coated in a conductive polymer polypyrrole. The reference electrode can be spaced apart from the ISFET by any suitable distance, e.g., 1, 2, 3, 4, 5, 6, 7, 8, 9, 10, 15, 20, 25, 30, 35, 40, 50 mm or more.

The support may also have a connector that can attach to electrochemical test strips configured for measurement of analytes of interest, e.g., luteinizing hormone, lactic acid, uric acid, glucose.

Devices and systems of the embodiments typically include subsystems including the ISFET, a reference electrode, a temperature sensor, a conductivity sensor, a readout circuit, and optionally, reference transistors and/or a heating element (e.g., to control temperature of the ISFET). The methods of the embodiments include signal processing methods, decision support methods, and user interface methods. Sensor placement and sample collection are also considerations. The devices and systems can be used as a component in a higher level system, often referred to as a health tracker, or simply a tracker for short. The health tracker includes as components the ISFET sensor module and associated signal processing methods, a processing system, data transmission systems (e.g., Bluetooth, serial port, 4G), mechanical components (straps, wristbands), and one or more user interfaces (display, buttons).

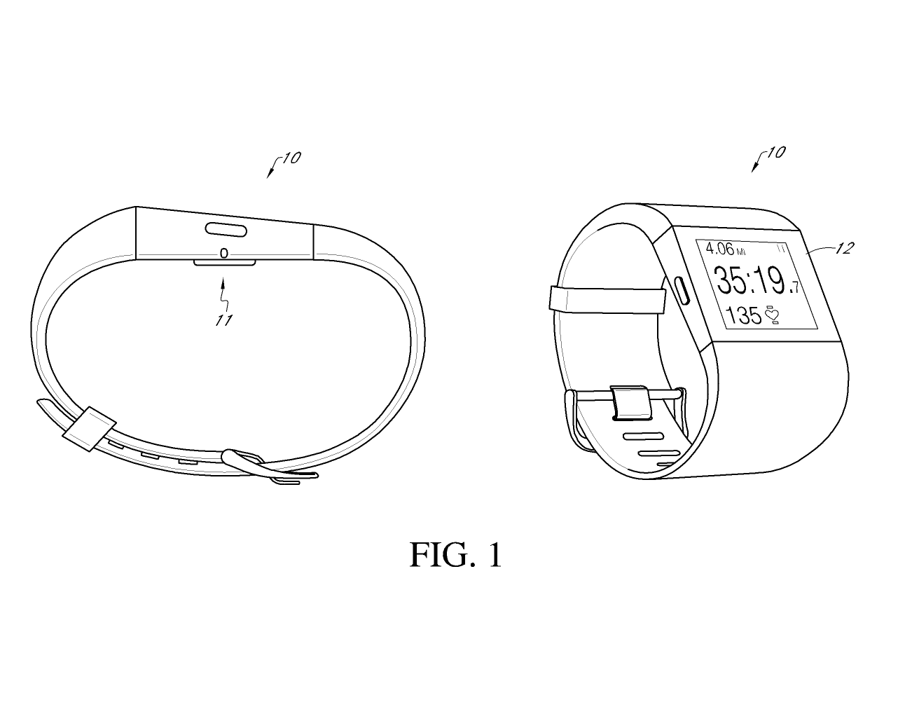

FIG. 1 is a rendering of a side view and a perspective view of a wearable device 10 that integrates an ISFET sensor. The wearable device 10 integrates an ISFET sensor on a protrusion 11, and the data from the wearable device 10 can be displayed on a user interface 12. In one embodiment, the ISFET is incorporated into a standalone sensing system, referred to as a ‘pebble’, which contains one or more sensors for human health and athletic performance monitoring. Software logic and/or algorithms are provided to implement methods for making the sensor data understandable. The pebble implementation and other embodiments as described herein are advantageous in that they facilitate the use of noninvasive wearable chemical sensors. Issues related to fragility, bulkiness, discomfort, skin irritation, imprecision, and signals that are hard to interpret are overcome by the devices of the embodiments.

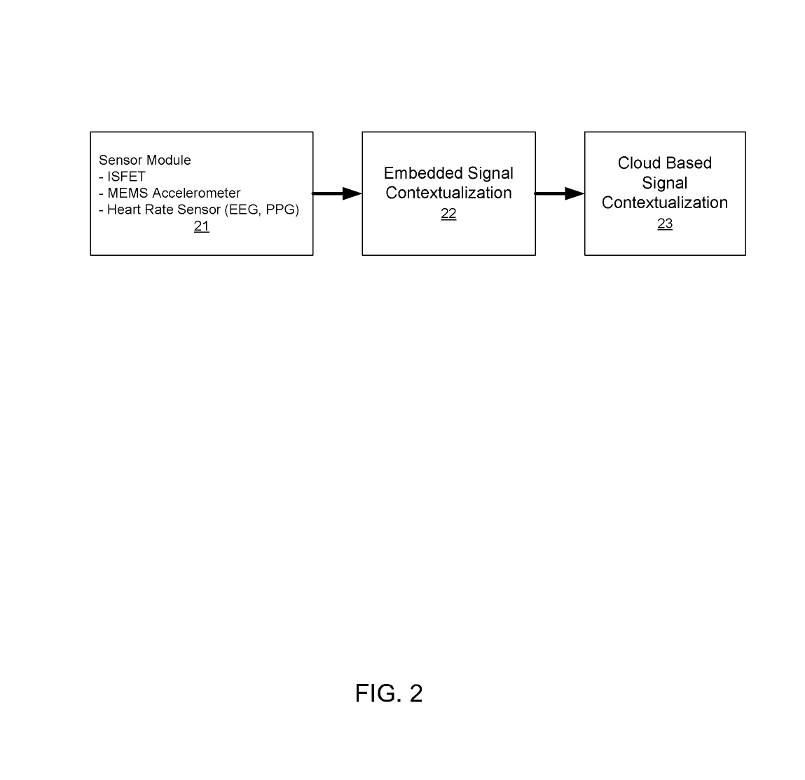

In one embodiment a removable/replaceable module that holds a set of ion selective field effect transistors is added to the skin-facing surface of a health tracker wristband, or integrated into an accessory wristband. This package of ISFETs can sit alongside any other optical or electronic sensors that may also face the skin. Signals from other sensors, most notably an accelerometer, a temperature sensor, and an optical heart rate sensor, may be employed in the processing and contextualization of signals from the sensor, as depicted in FIG. 2, which is a block diagram showing how signals from a sensor module 21 including an ISFET, MEMS accelerometer, and a heart rate sensor are employed in the processing and contextualization of embedded signals 22 from an ISFET sensor, and cloud based signal contextualization 23.

The devices and methods of the embodiments enable convenient measurement and/or monitoring of the pH and/or chemical composition of sweat and other body fluids originating from the skin or sweat. Some of these direct measurements may be used to indirectly estimate standard measures of endurance and athletic performance including lactate threshold, VO₂ max, and hydration status. References relating to sweat chloride levels include The Journal of Investigative Dermatology (1969) 53, 234-237; doi:10.1038/jid.1969.140; and JAMA. 1966; 195(8):629-635. doi:10.1001/jama.1966.03100080069018.

The data obtained can also be employed in the measurement of athletic endurance, measurement of skin acidity and sweat chemical composition during exercise, measurement of skin acidity and sweat chemical composition during and after the consumption of food, as an aid for maintaining appropriate hydration, and for the measurement of blood gas levels as an aid to athletic performance and relaxation exercises.

The ISFET devices of the embodiments overcome a number of disadvantages of other wearable devices. These disadvantages can include one or more of: avoidance of reference electrode materials that are vulnerable to damage by corrosion, avoidance of sensor materials that can cause skin irritation, avoidance of sensor materials that are fragile, avoidance of reference electrode materials that can cause skin irritation, avoidance of unconventional reference electrode materials that can decrease the precision of measurement, avoidance of electrode materials that are vulnerable to mechanical and moisture-related damage, avoidance of electrodes containing enzymes that can cause skin irritation and are vulnerable to degradation by enzymes, thermal denaturation, and chemical denaturation; avoidance of electrodes that are filled with liquid and are bulky, vulnerable to mechanical damage, and vulnerable to damage by contamination.

Wearable electrochemical sensors may be confounded by hand washing and other activities; however, as discussed herein, methods are provided for addressing certain of issues related to such activities.

The interpretation of wearable ISFET data may be challenging in the absence of non-chemical sensors that can contextualize the chemical sensor data, masking out data that are unfit for interpretation. Common activities including exercise, handwashing, and swimming can generate signals that must be dropped or contextualized in order to prevent confusion by the end user. Accordingly, as discussed herein, methods are provided for contextualizing the data.

Chemical sensors worn on the surface of the body must trap moisture from the skin without causing discomfort, without trapping air that may interfere with sensing, and must make consistent contact with the skin.

The devices and associated methods as provided herein accomplish one or more of these objectives.

### ISFET Sensor

ISFETs were first invented by Bergveld in the 1970s. Transistors have regions referred to as a source, drain, and gate. In a conventional transistor, the gate is completely covered by a packaging material or other sealing layer. In an ISFET, the gate is exposed to ambient. In operation of an ISFET, the current between the source and drain is modulated by the buildup of ions at the gate. ISFETs that measure sodium, potassium, chloride or other substances can be prepared by coating the gate with an ion-selective polymer membrane or by implanting ions directly into the gate. ISFETS are connected to a readout circuit. In general, the readout circuit is a feedback circuit that keeps the current through the transistor constant. From the readout circuit, the source-gate voltage is often passed to an analog to digital converter. This voltage is frequently the basis for calculations of ion concentration. Part of the readout circuit is a reference electrode. This electrode also comes into contact with the outside world. When measuring a specimen, both the gate of the transistor and the reference electrode are pressed against the specimen. In the case of this invention, the specimen is generally sweat, skin, and fluids that come from the skin. ISFET readout varies predictably with temperature, and commercially available ISFET sensor chips often contain a diode for temperature sensing.

ISFETs have been used to measure blood acidity and the levels of blood gasses since the 1990s. Several medical devices making use of ISFETs have been cleared for sale by the US Food and Drug Administration (FDA). Certain commercially available DNA sequencing instruments make use of ISFET arrays. The same class of sensors has also been applied to industrial process monitoring. Despite the long track record of use of these sensors in medical and industrial applications, no product on the market has heretofore made use of ISFETs in a wristband or garment meant for the monitoring of physically active people. For the most part, these sensors are meant for hours of use in contact with body fluids.

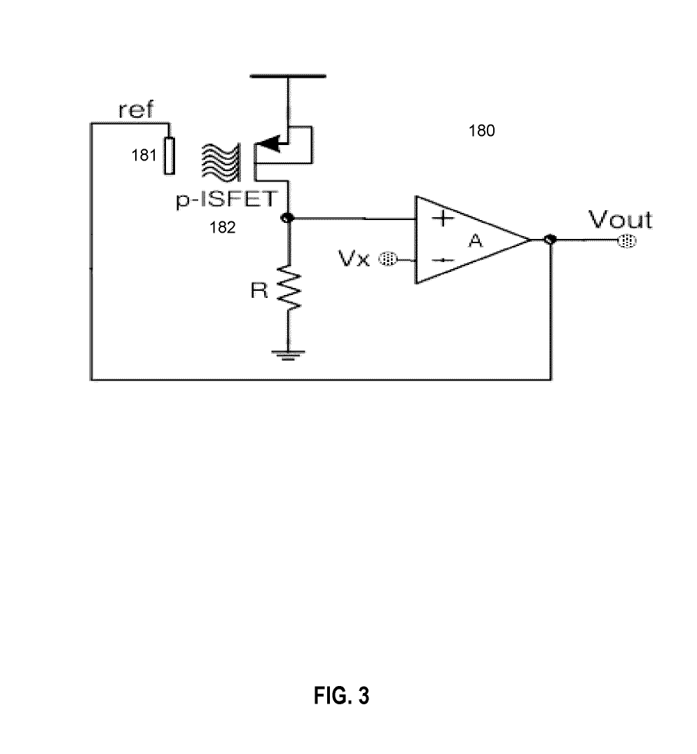

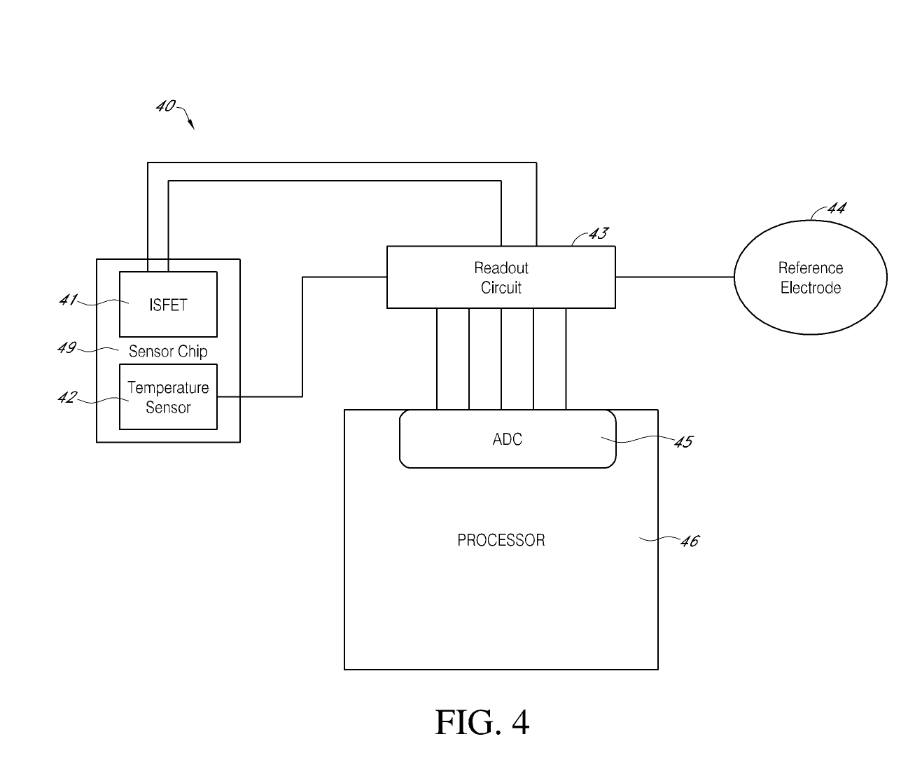

FIG. 3 is an electrical schematic depicting an exemplary ISFET sensor suitable for use in the wearable device in accordance with aspects of this disclosure. Exemplary ISFET(s) suitable for use as biosensors, e.g., pH sensors are described in Bergveld, P. and Sibbald, A., Analytical and biomedical applications of ion-selective field-effect transistors Comprehensive Analytical Chemistry, Eds.: Elsvier Amsterdam-Oxford-New York-Tokyo (1988), 172; Janata, J., Principles of Chemical Sensors in Modern Analytical Chemistry, Eds.: Plenum Press New York, London (1990), 317; and “ISFET, Theory and Practice”, Prof. Dr. Jr. P. Bergveld Em, IEEE Sensor Conference Toronto, October 2003, the contents of which is hereby incorporated by reference in its entirety. As shown in FIG. 3, an ISFET sensor 180 incorporates a reference electrode 181 and a p-ISFET 182. In an ISFET sensor, the voltage threshold changes in proportion to the concentration of H⁺ or other ions in the sample. The reference electrode acts as a gate. Readout circuits for ISFET sensors are known in the art. Many involve keeping the source-drain current constant with feedback from an op-amp. The ISFET sensor readout is generally calculated from Vₒᵤₜ, the source-reference voltage. The readout from an ISFET is often calculated from the source to reference potential. In the case of pH, assuming no temperature sensitivity and time drift:

$$pH = {pH}_{cal} + \frac{{V}_{gs}}{S}$$

where pH_{cal} is the pH of a calibration liquid at 37° C. (T_{cal}), V_{gs} the electrical output signal of the ISFET amplifier circuit and S the pH sensitivity (mV/pH) of the particular ISFET, stored in the memory of the device. ISFET(s) suitable for use in the wearable device 100 are available from a variety of manufacturers, including but not limited to Horiba, Shindengen, Microsens, Honeywell, Winsense, Sentron, MIMOS, ST Microelectronics, Cypress, Plessey Semiconductors, D+T Microelectronica, Freescal, Optoi, and SeaBird Scientific. FIG. 4. provides a schematic depicting a device 40 incorporating integration of an ISFET sensor 41 and temperature sensor 42 on a sensor chip 49 with a readout circuit, ADC processor, and reference electrode. The device includes a readout circuit 43, ADC 45 and processor 46, and reference electrode 44.

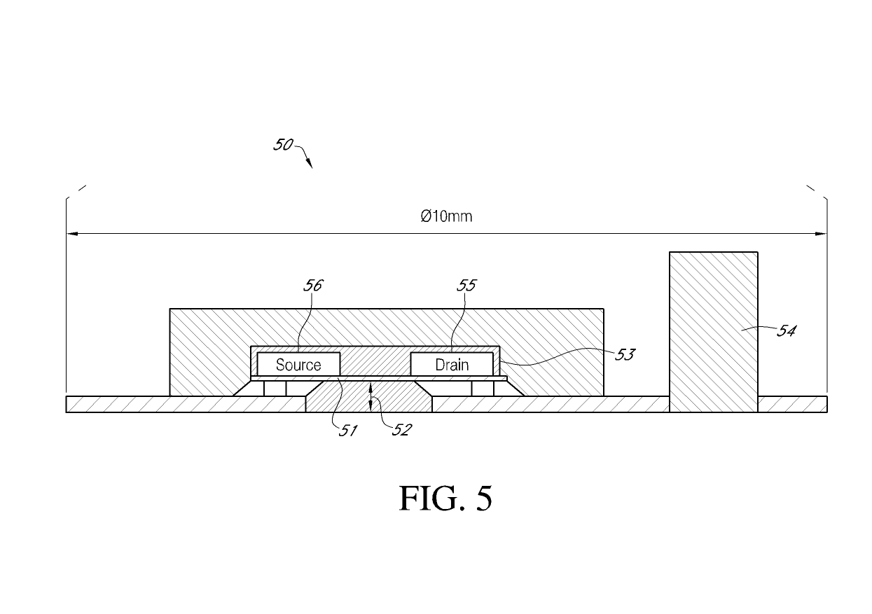

In one embodiment wherein skin pH is determined, a Horiba LAQUA™ pH sensor 50 can be employed, as depicted in FIG. 5. The sensor includes a source 56, a drain 55, a response membrane (gate) 51, a 100 micron gap 52 between the housing and the sensor, a silicon substrate 53, and a reference electrode 54. The response membrane is a glass membrane including a combination of rare earth metals to improve response time and to increase durability against chemical substances. A cation-conductive hollow fiber membrane covers the internal electrode, so as to minimize clogging by silver ions and silver complex ions from the Ag/AgCl reference electrode. The sensor is flat and very small in size, enabling the measurement of extremely small samples.

A wearable device comprising an ion selective field effect transistor in electrical communication with a reference electrode can advantageously be employed to measure characteristics associated with the presence of ions in a fluid at a user's skin surface. These characteristics include pH (as indicated by a concentration of hydrogen ions, H⁺) and concentration of ions such as H⁺ (reflected by pH) or Na⁺ and K⁺, each of which are components of eccrine sweat. Eccrine glands are the major sweat glands of the human body, found in virtually all skin. The highest density of these glands is in the palms and soles, followed by the head. Fewer of these glands are found on the trunk and the extremities. These glands produce a clear, odorless substance, consisting primarily of water and sodium chloride (NaCl), but also other electrolytes such as bicarbonate and potassium. Other components secreted in sweat include glucose, pyruvate, lactate, cytokines, immunoglobulins, antimicrobial peptides (e.g. dermcidin), and the like. These glands are active in thermoregulation by providing cooling from water evaporation of sweat secreted by the glands on the body surface.

The ISFET sensors employed in the wearable devices of the embodiments can detect the presence of ionic species, such as are present in fluids at the skin's surface. ISFETs can be configured (e.g., provided with ion selective membranes) to obtain selectivity to one particular ion, such as H⁺, Na⁺, or K⁺. An array of ISFETs can be provided, each configured to measure a particular ion. The wearable device can include one or more of a pH sensing ISFET, a Na⁺ sensing ISFET, and a K⁺ sensing ISFET. Other ions can also be sensed using ISFETs, including ammonium, calcium, magnesium, lead, nitrate, and chloride. The ISFET can be incorporated into any suitable portion of the wearable device, provided at least a portion of the ISFET is in contact with a skin surface. Advantageously, the ISFET is incorporated on the housing, described elsewhere herein, e.g., on a protrusion of the housing configured to enhance skin contact with the exposed portion of the ISFET.

The ISFET is employed in conjunction with a reference electrode (sometimes referred to as a pseudo-reference electrode). Any suitable reference electrode may be employed, including reference electrodes provided with (e.g., coated with) a permeable membrane. Conductive materials and systems suitable for use in the reference electrode include but are not limited to Ag/AgCl, polypyrrole, and poly(3,4-ethylenedioxythiophene). The reference electrode can comprise a conductive polymer material, either undoped or doped with one or more mediators, e.g., ferrocene and/or ferrocene derivatives. In some embodiments, the ISFET sensors employed in the devices and methods of the embodiments can be used in conjunction with metal-plastic composites or ceramic materials (e.g. Iridium Oxide) as a pseudo-reference electrode. In an embodiment, the reference electrode is made from a plastic film, ceramic substrate, or fabric coated with a conductive polymer such as polypyrrole. This material may have substantially higher durability than silver chloride or other classic reference electrode materials. Permeable membranes include but are not limited to membranes fabricated using, e.g., polyvinyl butyral or polyhydroxyethylmethacrylate. Representative examples include an Ag/AgCl electrode coated with a permeable membrane (e.g., polyvinyl butyral or polyhydroxyethylmethacrylate), an Ag/AgCl electrode coated with a permeable membrane (e.g., polyvinyl butyral or polyhydroxyethylmethacrylate) and saturated with chloride ions; a conductive polymer electrode (e.g., polypyrrole or poly(3,4-ethylenedioxythiophene)); a conductive polymer electrode (e.g., polypyrrole or poly(3,4-ethylenedioxythiophene)) mixed with or covalently bound to mediators such as ferrocene and its derivatives; a carbon paste electrode mixed with a mediator such as ferrocene or prussian blue and coated with a permeable membrane (e.g., polyvinyl butyral, nafion, or polyhydroxyethylmethacrylate); a noble metal reference electrode (e.g., gold, platinum) and/or a pseudo-reference electrode; and a noble metal reference (e.g., gold, platinum) and/or pseudo reference electrode paired with a reference field effect transistor. Other exemplary reference electrodes can include, but are not limited to, e.g., an Ag/AgCl electrode, an Ag/AgCl plastic composite electrode, an Ag/AgCl gel electrode, an Ag/AgCl electrode coated with a permeable membrane, a polypyrrole electrode, a poly(3,4-ethylenedioxythiophene) electrode.

In certain embodiments, an Ag/AgCl electrode configured for contact with skin can advantageously be employed. Such electrodes are commercially available as EMG/ECG/EKG self-adhering electrodes that employ an Ag/AgCl sensing element. Such electrodes typically include a snap connection, enabling the electrode to be placed in a convenient location on the body and a wired connection to the wearable device, e.g., via another snap connection, to be conveniently and readily established via an appropriate cable. As an alternative to a single or limited use disposable component as the reference electrode, a reference electrode can be built into the wearable device as a durable, reusable component. The reference electrode can be placed in proximity to the ISFET, e.g., on a protrusion on a housing of the wearable device, or on another portion of the wearable device that is in contact with a skin surface, e.g., the wristband or another portion of the housing other than a protrusion. Alternatively, the reference electrode can be integrated into an article of clothing worn against the skin, e.g., a wristband, a head band, a sports bra, a waistband, or other article of clothing in contact with the skin, and connected to the ISFET via a wired connection.

In addition to the ISFET and its associated reference electrode comprising the ISFET sensor, the wearable device advantageously includes a user interface, at least one processor, and a memory configured to analyze data from the ISFET sensor. The memory can store computer-executable instructions for controlling the at least one processor. With respect to the ISFET, the processor can be used to determine, based on output of the ion selective field effect transistor, at least one of pH and an ion concentration, and provide, via the user interface, information indicative of the pH or the ion concentration in a fluid at a surface of the user's skin.

The processor can be configured to sample the output of the ISFET in any suitable manner. This can include sampling at a constant rate, or at a variable rate, e.g., intermittently, either on a preselected schedule or at the initiation of another sensor incorporated into the wearable device or at the initiation of the user. Preferably, the processor is configured to sample the output of the ion selective field effect transistor at a faster rate when the user is physically active than when the user is sedentary (for example, as detected by sensors of the wearable device), e.g., for more accurate data during times of physical activity, and to conserve battery during times of inactivity. For example, the processor can be configured to sample the output at the faster rate when, e.g., initiated by the user, when a predetermined scheduled time arrives, immediately upon detection of exercise by sensors of the wearable device, or after the user has been physically active for at least a predetermined period, e.g., ten or more minutes.

Advantageously, the wearable device can further comprise a temperature sensor integrated with the ion selective field effect transistor. The temperature sensor can be configured to be in direct contact with the user's skin when the wearable device is in use, and can be used to measure skin surface temperature of the user as a physiologic metric. Alternatively, the temperature sensor can be configured to determine a temperature of the ISFET, and the temperature information employed to correct for any temperature effect on ion concentration data as determined by the ISFET. As an alternative to temperature correction, a heating element can be provided to maintain the ISFET sensor at a constant temperature, e.g., body temperature or a temperature above body temperature but within comfortable limits for exposure to the skin.

In operation, the ISFET sensor is configured to monitor a characteristic of a fluid at a surface of the user's skin, e.g., pH, electrolytic conductivity, Na⁺ concentration, or K⁺ concentration. The monitoring can be one or more of continuous or intermittent, long term or short term, or of constant rate or variable rate. Continuous sensing can be advantageously employed to track changes in an ion-related metric over time to determine trends, as described elsewhere herein. Intermittent sensing can be advantageous if an ion-related metric is of interest in conjunction with, e.g., exercise activity or to troubleshoot an issue (e.g., residue buildup on the wearable device). Multiple ISFET sensors, each operating independently or in conjunction, can be employed to monitor multiple ion-related metrics, or the same metric so as to provide an accuracy check by comparing data from each sensor.

In operation the specimen (sweat and other skin fluids) gets onto the ISFET sensing element through direct contact of the ISFET with skin. Commercially available ISFETs typically are fabricated from components that resist damage by oxidation or reduction, e.g., insulating polymeric materials, glass, or nonconductive composite materials. The ISFET sensing element makes use of an optical, electronic, or mechanical readout provided on the wearable device or by an auxiliary device (e.g., smartphone, tablet, computer). For detecting certain chemical species, the ISFET may be accessorized with enzymes or small molecules, as discussed herein, or may not require such auxiliary materials in order to detect an analyte of interest. ISFETs as described herein typically do not result in any user-detectable chemical reactions at the sensing element, or electrical activity at the sensor element causing bubbles to form within the specimen, and are generally robust such that the materials in the sensing element are not vulnerable to cracking, scratches or dissolution during normal use and wear.

In preferred embodiments, the ISFET sensor is designed in such a way that it rarely becomes dry. This can be accomplished by providing the ISFET with a moisture trapping means. For example, the protrusion upon which the ISFET sits may incorporate a concave region configured to trap moisture thereunder. The protrusion may incorporate an elastomeric material surrounding the ISFET, to form a seal that traps moisture. A moisture absorbing material (polymeric sponge, water absorbing membrane, or the like) can be provided in proximity to the ISFET, e.g., as an annular structure, or an adjacent structure of suitable size and shape. Sensor regeneration and movement of old specimen fluid away from the sensor and the movement of new specimen fluid toward the sensor can be facilitated by motion of the sensor during activity. The devices of the embodiments that employ a protruding area, referred to as a ‘protrusion’ upon which at least a portion of the ISFET is situated ensure good contact between the sensor and the skin, and concentrate moisture from the skin around the sensor. Sensing elements that must be in contact with the skin are confined to a protruding part of the invention. The protrusion can be designed to ensure contact between the skin and the sensing elements. In one embodiment, the protrusion has a convex surface, and in another embodiment it has a shallow concave surface to enhance the trapping of moisture. Under most circumstances, the skin slowly vents moisture. When the sensor surface is made from a glassy material, an adequate amount of moisture can accumulate on the sensor surface. Even if skin feels dry to the touch, moisture buildup will quickly occur if the sensor is pressed firmly against the skin.

In one embodiment, the ISFET sensor is incorporated on a rear face of a fitness tracker, adjacent to an optical sensor. In another embodiment, the ISFET sensor is positioned on the rear face of a fitness tracker, adjacent to galvanic skin response sensor. The sensor protrudes slightly from the body of the fitness tracker, e.g., 0.5 mm or less to 2 mm or more, so as to provide better skin contact. A rim surrounding the sensing elements aids in the trapping of moisture. The rim can be unitary with the housing, or of a different material. The ISFET can be provided in a form of a skin facing surface of a capsule configured for insertion into specially designed athletic clothing.

The associated software and data from accessory sensors can be used to contextualize and filter the readout of the ISFET sensor, including use of signal processing and estimation techniques to make the sensor output easily understandable by the end user. Specific signal processing techniques used in the devices and methods of the embodiments include: making multiple measurements per minute; smoothing with a rolling median; masking out of data when the sensor is ‘off-wrist’ as determined by other sensors; masking out of data when the sensor signal is rapidly changing; masking out of data when the sensor circuit is open; flagging of data during periods when the sensor temperature is changing rapidly; flagging of data during periods when an accelerometer indicates sedentary or sleep state; and flagging of data during periods when a heart rate sensor or accelerometer indicates exercise. In certain embodiments, anaerobic threshold measurements are made when motion sensors indicate that the device is in use by a person who is currently exercising.

### Monitoring Skin Health

A wearable device incorporating an ISFET sensor can be employed for monitoring skin health. One such method involves using the ISFET sensor to measure a pH of a fluid at the user's skin surface. Skin has evolved to fight infection and environmental stresses, and its ability to do so is affected by its pH level. Skin pH levels are discussed in Schmid-Wendtner et al. “The pH of the Skin Surface and Its Impact on the Barrier Function”, Skin Pharmacol. Physiol. 2006; 19:296-302; and Lambers et al. “Natural skin surface pH is on average below 5, which is beneficial for its resident flora”, Int. J. Cosmet. Sci. 2006 October; 28(5):359-70. Skin has a thin, protective layer on its surface, referred to as the acid mantle. This acid mantle is made up of sebum (comprising free fatty acids) excreted from the skin's sebaceous glands. Sebum mixes with lactic and amino acids from sweat, which determines the skin's pH. For healthy skin, the pH should be slightly acidic at about 5.5. Many factors can interfere with the function of the skin's acid mantle, both externally and internally. As skin ages, it typically becomes more acidic in response to lifestyle and environmental factors, including diet, exercise, the use of skin products, smoking, air quality, water quality, exposure to sun, and exposure to environmental pollutants. These exposures can contribute to the breaking down of the acid mantle, disrupting the skin's ability to protect itself. A pH level that is too alkaline or too acidic indicates that the acid mantle may be disturbed and can be associated with skin conditions such as dermatitis, eczema, and rosacea. Many cleansers, including bars and detergent soaps, tend to be alkaline and act to remove natural oils from the skin surface, causing dryness and irritation. Skin that is too alkaline can be more susceptible to acne because a certain level of acidity is needed to inhibit bacterial growth on the skin. Conversely, exposure of the skin to products that are overly acidic can also be problematic. Such products can also remove natural oils, which can temporarily disrupt the lipid barrier of the skin.

The ISFET sensor can be employed to determine if the pH is outside of a range indicative of good health. Variable skin pH values have been reported in literature, all in the acidic range but with a broad range from pH 4.0 to 7.0. The ISFET can determine if the pH falls within a narrow range around optimal skin pH, e.g., a pH within a range of 5 to 6 is indicative of good skin health, while a value of 4 to 5 or 6 to 7 may be indicative of skin irritation or poor skin health, or the presence of alkaline or acidic residues on the skin, e.g., from skin care products (alkaline cleansers, acidic medicinal creams) or exposure to seawater (typically of pH of 7.5 to 8.4). Values outside of the range of physiological values may indicate the presence of residues from skin care products or cleansers, exposure to liquids such as sea water, or other acidic or alkaline substances in the environment. The threshold pH or pH range(s) used for determining that the skin may not be in a healthy pH range may be predetermined, e.g., set by an algorithm and/or logic on the wearable device or a server (e.g., a cloud software service) in communication with the wearable device, or can alternatively be set by the user, taking into consideration the user's unique physical characteristics. In some embodiments, the ISFET sensor can be employed to measure a user's pH, e.g., at rest, during exercise, or the like, and this value stored for reference by an algorithm and/or logic on the wearable device to set one or more customized ranges. Different ranges may be employed, e.g., one range for rest, one for exercise, or the like. The data obtained from the ISFET sensor can then be analyzed to output information to the user from the wearable device. This information can be as simple or detailed as desired. For example, the information can be as simple as an indication of state of the skin (“Healthy” or “Possible Skin Irritation or Poor Skin Health” displayed as text, or a green versus a red symbol or text tagged to “Skin Health”). Alternatively, a pH value can be output, e.g., the last pH value measured, an average pH value calculated from a collection of data points obtained over a fixed period of time, or a moving average pH. The pH can be displayed in relation to a user's normal pH or a theoretical optimal pH. The pH data can be stored and analyzed to identify trends. In certain embodiments, the ISFET sensor may continuously or intermittently monitor pH levels, and the wearable device can issue an alert if pH values outside of a preselected range are measured, or the wearable device can output the information associated with pH at predetermined times. Alternatively, the user can query the wearable device to determine a current pH, or information from past pH measurements.

In conjunction with the ISFET sensor, an optical sensor, such as a UV sensor can be employed to obtain data that may be indicative of health of a user's skin. One embodiment involves determining an initial UV absorption value for the user's skin, which is stored by the wearable device. Future UV absorption values can be measured and compared against the initial value to determine if a change in skin condition has occurred. Such data can also be used to corroborate data from the ISFET sensor. For example, skin discoloration (e.g., reddening) may occur if skin is irritated or damaged (e.g., by sunburn). The presence of a change in UV absorption value coupled with an elevated pH level may provide a stronger indication of skin health. This information can then be provided via the wearable device (e.g., an output of “Possible Skin Irritation” versus “Skin Irritation Detected”). An algorithm and/or logic of the wearable device can be employed to analyze data from the ISFET and optical sensor to determine an appropriate information output, which may also be no output at all if no potential skin issues are detected.

As discussed above, information indicative of skin health can be output on a user interface associated with the wearable device or another computing or another device (a client device) in communication (wired or wireless) with the wearable device. The user interface can include at least one of a display, a light-emitting circuit, a sound-producing circuit, and a haptic drive circuit. Advantageously, the wearable device can comprise a transceiver configured to communicate with a client device, e.g., a personal computer, a mobile phone, or a tablet computing device.

### Monitoring the Presence of Residue Buildup

As discussed above, measurement of pH of a fluid at the skin surface can provide data indicative of skin health. Similarly, such pH measurement can also provide data indicative of residue buildup on a surface of the wearable device or an associated garment. Such residue buildup can include soap residue buildup, grease residue buildup, skin cream residue buildup, and sunblock residue buildup. Residue buildup can cause the measured pH to be outside of the physiological range, e.g., greater than 7 or less than 4. Alternatively, the residue buildup may result in high variability of ion-related metric measurements (e.g., substantial differences in adjacent data points or adjacent data points that are physiologically impossible), or even the inability to sense pH at all, if conductivity to the ISFET is blocked by an insulating greasy layer. Whether or not the degree of variability in the data falls within an acceptable range can be determined by comparing data against a stored set of criteria, or by comparison to representative data that was previously collected and stored for the user.

Buildup of soap or cleanser residue on the device may present issues. These residues are often highly alkaline in nature, or contain high amounts of sodium or potassium counter ions.

In operation, the ISFET is employed to measure a characteristic of a fluid present on a user's skin and then the processor determines, based on the measured characteristic of the fluid, an amount of buildup on the wearable device or the garment exceeding a threshold amount. Information can then be provided, via the user interface, indicative of a cleanliness of the wearable device or the garment. If the characteristic is pH, then measurement of a pH greater than 7 (e.g., 7.5 or even 8, 8.5, 9, 9.5, or 10 or higher) by the ISFET sensor may indicate the presence of residue buildup. If the ion-related metric is an ion concentration, then a measurement falling outside of the physiological range may indicate residue buildup, e.g., an ion concentration (e.g., sodium ion or potassium ion) greater than 1.1 times a physiological maximum ion concentration in eccrine sweat indicates residue buildup. As in the case of pH, high variability of measurements or inability to sense ions may also indicate residue buildup.

### Monitoring a State of Hydration

An ISFET sensor, or combination of ISFET sensors, can be provided that can measure an output reflective of overall ion concentration of a fluid at a surface of a user's skin. This data can be used to determine a state of hydration of the user, and, if desired providing, via the user interface, information indicative of the user's state of hydration. A dedicated subroutine for dehydration detection can advantageously be activated by a period of physical activity. As with the other ion-related metrics, the wearable device or an associated client device or server can provide standard ion-related metrics indicative of a normal state of hydration against which the data obtained from the ISFET can be compared. Alternatively, the user's own data obtained in a hydrated state can be employed as reference data. When a dehydrated state is occurred, e.g., as indicated by overall ion concentration exceeding a threshold, an alert can be provided via the user interface. Buildup of certain residues may also result in elevated overall ion concentration; however, residue buildup is often associated with non-physiological pH. Data from an ISFET sensor measuring pH can be compared against the overall ion concentration data from one or more other ISFET sensors. If a physiological pH is detected in connection with elevated overall ion concentration, then a dehydrated state may be more likely than if a nonphysiological pH is present along with an elevated overall ion concentration.

### Additional Sensors

In certain embodiments, the wearable device advantageously incorporates one or more additional biometric sensors in addition to the ISFET sensor. When additional biometric sensors are present, these can operate independently from the ISFET sensor, or can operate in conjunction with the ISFET sensor. One advantageous method for operation is to use one or more biometric sensors to obtain one or more user physiological metrics. These metrics can then be employed to improve an accuracy of the information provided via the user interface related to the ISFET sensor. For example, detection of exercise may be used to initiate a faster sampling rate for the ISFET sensor so as to better reflect changing ion concentrations during exercise. Alternatively, a reduced sampling rate can be initiated upon, e.g., resting or sleeping, so as to conserve battery power.

As described herein, additional physiological metrics can be measured and can be used in conjunction with the pH or ion measurements obtained by the ISFET sensor in the wearable device. These metrics can include, but are not limited to, user heart rate, user photoplethysmography, user blood pressure, user respiration rate, user skin conduction, user blood glucose levels, user blood oxygenation, user skin temperature, user body temperature, user electromyography, and user electroencephalography. Of interest for fitness tracking are CO₂ chemical sensors and heart rate sensors integrated into the wearable device to detect anaerobic threshold if heart rate is in exercise zones. Capacitance sensors integrated into the wearable device may be useful in identifying measurements made on dry skin, or measurements made when the device is not in contact with skin, or when the device is immersed in water. A glucose sensor can be integrated into the wearable device, the glucose sensor being configured for use in conjunction with an electrochemical test strip. Similarly, environmental metrics can be measured and can be used in conjunction with the pH or ion measurements obtained by the ISFET sensor in the wearable device. These can include ambient temperature, ambient humidity, geolocation, motion, time of day, date, or the like. Alternatively, or in addition to measured metrics, the wearable device can accept user inputs or inputs from another source. The user inputs can be self-reported level of activity, physiological condition, or the like. These metrics or user inputs can be employed to detect a condition wherein a faster or slower data sampling rate of the ISFET sensor or another biometric sensor can be initiated, e.g., detection of exercise, or to initiate or cease sampling of data by a sensor such as the ISFET sensor, and/or the output or storage of information related to a measured metric, as described herein.

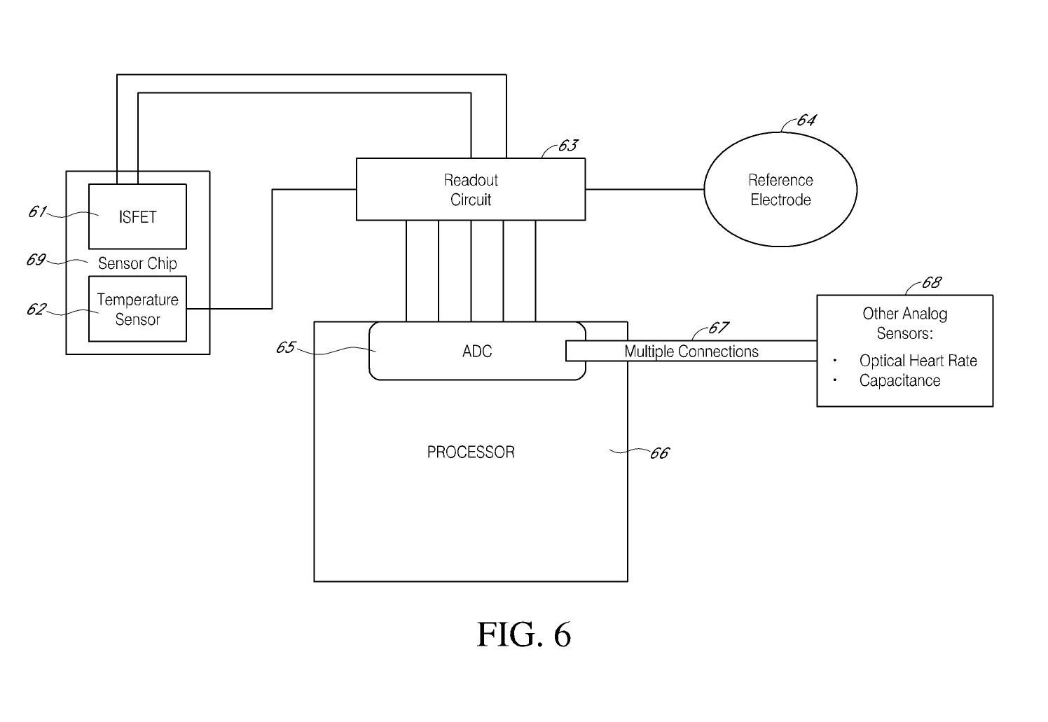

FIG. 6 provides a schematic depicting integration of an ISFET sensor 61 and temperature sensor 62 on a sensor chip 69 with a readout circuit 63, ADC 65 and processor 66, and reference electrode 64, wherein analog sensors 68 including an optical heart rate sensor and a capacitance sensor are connected to the ADC via multiple connections 67.

### Health Tracker Incorporating ISFET

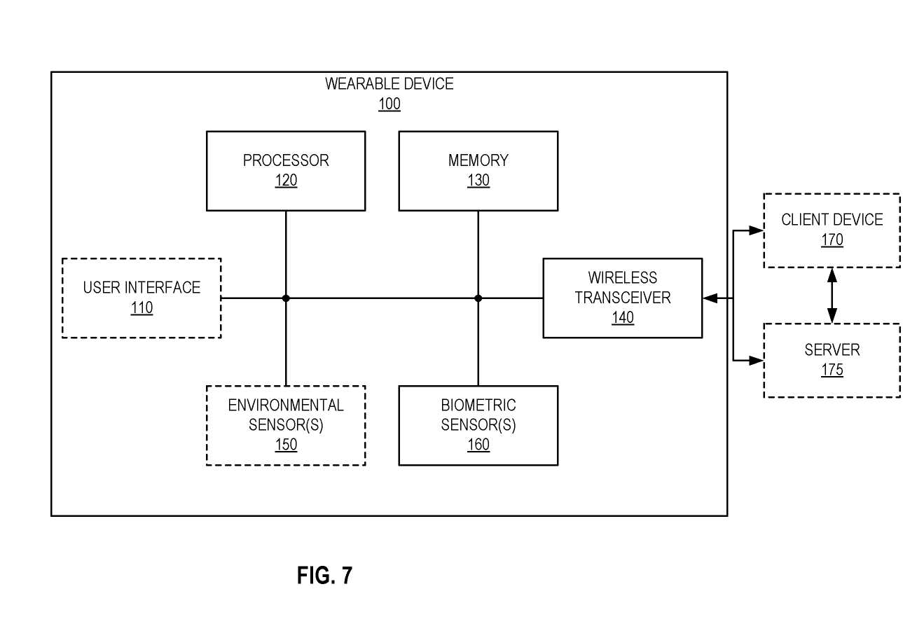

FIG. 7 is a block diagram illustrating an example wearable device in accordance with aspects of this disclosure. The wearable device 100 may include a processor 120, a memory 130, a wireless transceiver 140, and one or more biometric sensor(s) 160, e.g., ISFET(s) as described herein. The wearable device 100 may also optionally include a user interface 110 and one or more environmental sensor(s) 150. The wireless transceiver 140 may be configured to wirelessly communicate with a client device 170 and/or server 175, for example, either directly or when in range of a wireless access point (not illustrated) (e.g., via a personal area network (PAN) such as Bluetooth pairing, via a WLAN, etc.). Each of the memory 130, the wireless transceiver 140, the one or more biometric sensor(s) 160, the user interface 110, and/or the one or more environmental sensor(s) 150 may be in electrical communication with the processor 120.

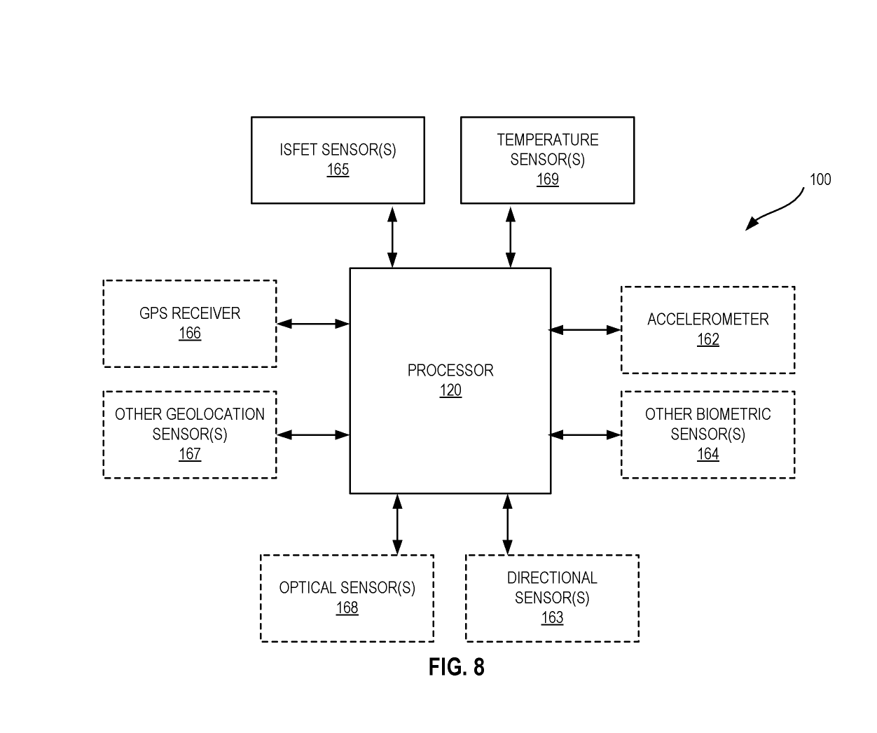

The memory 130 may store instructions for causing the processor 120 to perform certain actions. For example, the processor 120 may be configured to automatically detect the start of an exercise performed by a user of the wearable device 100, a state of exertion of the user, or an environmental condition and adjust a sampling rate for the ISFET based on instructions stored in the memory 130. The processor 120 may receive input from the one or more of the biometric sensor(s) 160, e.g., the ISFET(s) and/or the one or more environmental sensor(s) 150 in order to determine a state of exertion of the user or an environmental condition. In some embodiments, the biometric sensors 160 may include, in addition to the ISFET(s), one or more of an optical sensor (e.g., a photoplethysmographic (PPG) sensor, an optical heart rate sensor), an accelerometer (e.g., a MEMS accelerometer), a GPS receiver, a temperature sensor, galvanic skin response circuit, a moisture sensor, and/or other biometric sensor(s). Further information regarding such biometric sensors is described in more detail below (e.g., in connection with FIG. 8). Data from one or more of the other biometric sensors, e.g., an accelerometer or photoplethysmograph, can be used for the purpose of filtering data from an ISFET sensor.

The wearable device 100 may collect one or more types of physiological and/or environmental data from the one or more biometric sensor(s) 160, the one or more environmental sensor(s) 150, and/or external devices and communicate or relay such information to other devices (e.g., the client device 170 and/or the server 175), thus permitting the collected data to be viewed, for example, using a web browser or network-based application. For example, while being worn by the user, the wearable device 100 may perform biometric monitoring of pH and/or ion levels in a fluid at a skin surface using the one or more biometric sensor(s) 160. The wearable device 100 may transmit data representative of the pH and/or ion levels to an account on a web service (e.g., www.fitbit.com), computer, mobile phone, and/or health station where the data may be stored, processed, and/or visualized by the user. The wearable device 100 may measure or calculate other physiological metric(s) in addition to, or in place of, the user's pH and/or ion levels. Such physiological metric(s) may include, but are not limited to: step count, energy expenditure, e.g., calorie burn; floors climbed and/or descended; heart rate; heartbeat waveform; heart rate variability; heart rate recovery; location and/or heading (e.g., via a GPS, global navigation satellite system (GLONASS), or a similar system); elevation; ambulatory speed and/or distance traveled; swimming lap count; swimming stroke type and count detected; bicycle distance and/or speed; blood pressure; blood glucose; skin conduction; skin and/or body temperature; muscle state measured via electromyography; brain activity as measured by electroencephalography; weight; body fat; caloric intake; nutritional intake from food; medication intake; sleep periods (e.g., clock time, sleep phases, sleep quality and/or duration); pH levels; hydration levels; respiration rate; and/or other physiological metrics.

The wearable device 100 may also measure or calculate metrics related to the environment around the user (e.g., with the one or more environmental sensor(s) 150), such as, for example, barometric pressure, weather conditions (e.g., temperature, humidity, pollen count, air quality, rain/snow conditions, wind speed), light exposure (e.g., ambient light, ultra-violet (UV) light exposure, time and/or duration spent in darkness), noise exposure, radiation exposure, and/or magnetic field. Furthermore, the wearable device 100 (and/or the client device 170 and/or the server 175) may collect data from the biometric sensor(s) 160 and/or the environmental sensor(s) 150, and may calculate metrics derived from such data. For example, the wearable device 100 (and/or the client device 170 and/or the server 175) may calculate the user's stress or relaxation levels based on a combination of heart rate variability, skin conduction, noise pollution, and/or sleep quality. In another example, the wearable device 100 (and/or the client device 170 and/or the server 175) may determine the efficacy of a medical intervention, for example, medication, based on a combination of data relating to medication intake, sleep, and/or activity. In yet another example, the wearable device 100 (and/or the client device 170 and/or the server 22) may determine the efficacy of an allergy medication based on a combination of data relating to pollen levels, medication intake, sleep and/or activity. These examples are provided for illustration only and are not intended to be limiting or exhaustive.

FIG. 8 is a block diagram illustrating a number of example biometric sensors that may be in communication with the processor of the wearable device in accordance with aspects of this disclosure. For example, in the embodiment of FIG. 8, includes one or more ISFET sensor(s) 165. The wearable device 100 may optionally include temperature sensor(s) 169 which may be used to determine ambient temperature or a temperature of user's skin. The wearable device 100 may optionally include a GPS receiver 166 which may be used to determine the geolocation of the wearable device 100. The wearable device 100 may further include optional geolocation sensor(s) 167 (e.g., WWAN and/or WLAN radio component(s)), in addition to or in lieu of the optional GPS receiver 166. The wearable device 100 may further include optional optical sensor(s) 168 (e.g., a PPG sensor), and may optionally include an accelerometer 162 (e.g., a step counter), direction sensor(s) 163, and/or other biometric sensor(s) 164. Examples of the directional sensor(s) include the accelerometer 162, gyroscopes, magnetometers, etc. Each of the biometric sensors illustrated in FIG. 8 is in electrical communication with the processor 120. The processor 120 may use input received from any combination of the GPS receiver 166, the optical sensor(s) 168, the accelerometer 162, and/or the other biometric sensor(s) 164 in detecting the start of an exercise and/or in tracking the exercise. In some embodiments, the GPS receiver 166, the optical sensor(s) 168, the accelerometer 162, and/or the other biometric sensor(s) 164 may also correspond to the biometric sensor(s) 160 illustrated in FIG. 7.

In one embodiment of a system, a wearable device is provided that contains multiple sensors. Each of these sensors monitors a different physiological signal. Data collected from the sensors are interpreted by an algorithm, which provides the user with metrics related to circadian rhythm, stress level, sweat pH, and sweat ion concentrations. Sensor types that may be included in this system are: one or more ISFETs, one or more ion specific electrodes, an optical sensor capable of estimating heart rate, an optical sensor capable of measuring hemoglobin levels, a temperature sensor for measuring skin temperature, an electronic sensor capable of measuring electrocardiogram type signals, and an electronic sensor capable of measuring sweat and skin conductivity. The device can measure a single metric or multiple metrics, simultaneously or sequentially, e.g., pH of sweat and other fluids from the skin, sodium, potassium, magnesium, chloride, ammonium, phosphate, oxygen, carbon dioxide, and/or calcium.

In a preferred configuration, the wearable device is worn like a wristwatch on the wrist. In alternate configurations, it is held against the body by a shirt, pants, or other garment, worn on a necklace around the neck, worn on the head in the rim of a hat, or held to the skin with an adhesive strip.

It related aspects, the processor 120 and other component(s) of the wearable device 100 (e.g., shown in FIG. 7 and FIG. 8) may be implemented as any of a variety of suitable circuitry, such as one or more microprocessors, application specific integrated circuits (ASICs), field programmable gate arrays (FPGAs), discrete logic, software, hardware, firmware or any combinations thereof. When the techniques are implemented partially in software, a device may store instructions for the software in a suitable, non-transitory computer-readable medium and execute the instructions in hardware using one or more processors to perform the techniques of this disclosure.

In further related aspects, the processor 120 and other component(s) of the wearable device 100 may be implemented as a System-on-a-Chip (SoC), that may include one or more CPU cores that use, e.g., one or more reduced instruction set computing (RISC) instruction sets, a GPS receiver 166, a WWAN radio circuit, a WLAN radio circuit, and/or other software and hardware to support the wearable device 100.

The signal output generated by the ISFET sensor can be analyzed or processed using a processor, memory, and associated algorithms and/or logic. This analysis and processing can include one or more of the following: signals collected in a time period following sensor activation are flagged as garbage or discarded; signals outside of the plausible physiological range are flagged as environmental data or dropped; signals from a companion sensor that measures capacitance, optical heart rate, motion, or galvanic skin response may be used to detect that the sensor is not in contact with the skin; sudden changes in sensor readings are flagged; sensor readings matching the parameters of ocean water are tagged as such; sensor readings matching the parameters of pool water are tagged as such; sensor readings matching the parameters of a shower or bath are tagged as such; sensor readings consistent with oxygen desaturation are tagged as such; sensor readings consistent with uncomfortably low body pH (acidosis) are tagged as such; sensor readings in the absence of a detectable heart rate are flagged as garbage or discarded; sensor readings collected during a period when the rate of temperature change exceeds a threshold are discarded or flagged as environmental data; sensor readings collected during a period of high skin conductance are flagged as such; and sensor readings collected during a period of low skin conductance are flagged as such.

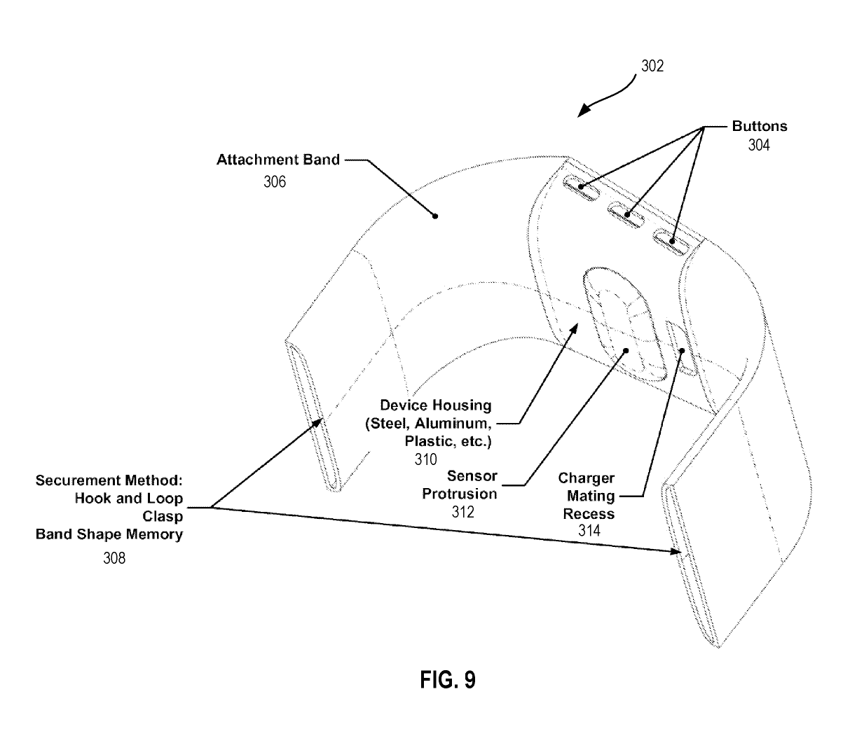

FIG. 9 provides a diagram illustrating a wearable device 302 of one embodiment including an ion selective field effect transistor (ISFET) electrically coupled to a reference electrode. The device includes an attachment band 306, buttons for control of various features of wearable device 302, a device housing 310 (e.g., steel, aluminum, plastic, or other suitable material), a charger mating recess 314, a securement method 308 (e.g., a hook and loop, a clasp, or a band shape memory), and a sensor protrusion 312. The ISFET and/or reference electrode (and/or component(s) of the ISFET and/or the reference electrode) can advantageously be situated on the sensor protrusion 312, the device housing 310, the attachment band 306, and/or any other suitable location in contact with a user's skin.

A device of any suitable configuration can be employed to retain the ISFET sensor. For example, a wristwatch type device incorporating an ISFET sensor module that measures the pH of sweat and other fluids from the skin, along with a sensor that measures skin temperature can be provided. Other subsystems of the wristwatch measure heart rate, heart rate variability, galvanic skin response, motion, and oxygen saturation.

A rugged capsule can be provided that can be inserted into a pouch within athletic clothing, where the ISFET sensor module is held firmly against the skin or against an area of clothing that often becomes soaked with sweat during exercise. The ISFET sensor module measures the pH of sweat and other fluids from the skin, along with a sensor that measures skin temperature.

A wristwatch type device incorporating an ISFET sensor array that measures the pH of sweat and other fluids from the skin, a grid of sensors that measure electrolytes (optionally magnesium, potassium, chloride, sodium) along with a sensor that measures skin temperature can be employed. Other subsystems of the wristwatch measure heart rate, heart rate variability, motion, and oxygen saturation.

A rugged capsule that can be inserted into a pouch within athletic clothing is advantageous, where the sensor module is held firmly against the skin. The ISFET sensor array measures the pH of sweat and other fluids from the skin, a grid of other sensors, e.g., ISFET sensors, can be provided that measure electrolytes (optionally magnesium, potassium, chloride, sodium) along with a sensor that measures skin temperature.

These devices offer one or more advantages in terms of durability, biocompatibility, precision, contextualization, ease of use, and end user guidance. Unlike other wearable sensors, the ISFET chip is made from ceramic materials, which are highly resistant to chemical and mechanical damage. Furthermore, the sensor chip is mounted into the system in a manner that minimizes the risk that it will break under tension when placed under pressure. A facet of the use of ISFET sensors is the avoidance of enzymes, plasticizers, mediators and water-soluble materials that may make contact with the skin. Measurements from the ISFET can be conducted in parallel with measurements from auxiliary sensors, including an optical heart rate sensor (photoplethysmography), a temperature sensor, and an accelerometer. Data from these auxiliary sensors can be used to identify chemical sensor signals that should be discarded without storage or interpretation. The devices compensate for imprecision created through the use of a durable pseudo-reference electrode, and also compensates for signal drift caused by long term wear. While many wearable devices that continuously measure body chemistry are available, the devices of the embodiments are heretofore the only wearable devices that place an extraordinarily durable sensor in contact with the skin without breaking the skin, in contrast to conventional devices incorporating fragile electrode materials, sensors that penetrate the skin, or sensors that include an enzyme entrapped beneath a membrane. Sensing by the device of the embodiments can be automatically activated and deactivated by software, without any need for intervention by the end user, and the device can contextualize chemical sensor data and masks out data unfit for interpretation.

The devices of the embodiments are suitable for monitoring a variety of physiological conditions. Serum pH levels are known to decrease during intense exercise. Sweat pH also is known to decrease in step with exercise, but it does not decrease in step with serum. During periods of intense exercise, the accelerometer sensor of the device can detect high levels of motion and increases the sampling rate of the chemical sensor package. Data collected during intense exercise may be automatically analyzed against heart rate, GPS data, and accelerometer data. The resulting output to the end user may include estimates of VO₂ max, lactate threshold, and other established measures of endurance.

The device of the embodiments can include methods for alerting the user when the sensor module must be cleaned or replaced. When measurements from the sensor fall outside of an expected range, an alert to clean the sensor is communicated to the end user. When changes in the sensor signal stray from an expected time series, an alert to clean the sensor is communicated to the end user. When a sudden change in the sensor signal is not accompanied by signs of exercise (an increase in heart rate and motion) the user is instructed to clean the sensor.

The device of the embodiments outputs sensor data in a format suitable for processing and temporary storage on an embedded device. The device can output sensor data in a format suitable for transfer to mobile devices and the cloud via low bandwidth connections. Although the sampling rate of the ISFET sensor can be very high, data can be saved at intervals that create log files of manageable size. The device can draw from multiple sensors to provide higher accuracy.

### ISFET Sensor Operation by a User

### Example A

Sally the user wears a device comprising a wristwatch containing the ISFET sensor on her non-dominant arm. The device periodically evaluates whether it should be making measurements. Moisture is trapped between the invention and Sally's skin. The level of moisture trapped between the ISFET sensor and Sally's skin increases to a critical point. The ISFET sensor begins periodically measuring the pH of the fluid on the ISFET sensor surface. These pH data are stored in the device. Sally washes her hands, and some soap water gets onto the sensor surface. The ISFET sensor detects a sharp increase in alkalinity and sends a message to the device. The device instructs Sally to clean and dry the sensor surface. Sally cleans the sensor and pH logging resumes. Sally goes for a swim in the ocean. The temperature sensor notes a sudden drop in temperature. The device flags measurements made during the swim as environmental logs. Sally goes for a run, and the frequency of sensor measurements is increased when the accelerometer or optical heart rate sensor detects an increase in movement and heart rate.

### Determining Cleanliness of Wearable Device or Associated Garment

### Example B

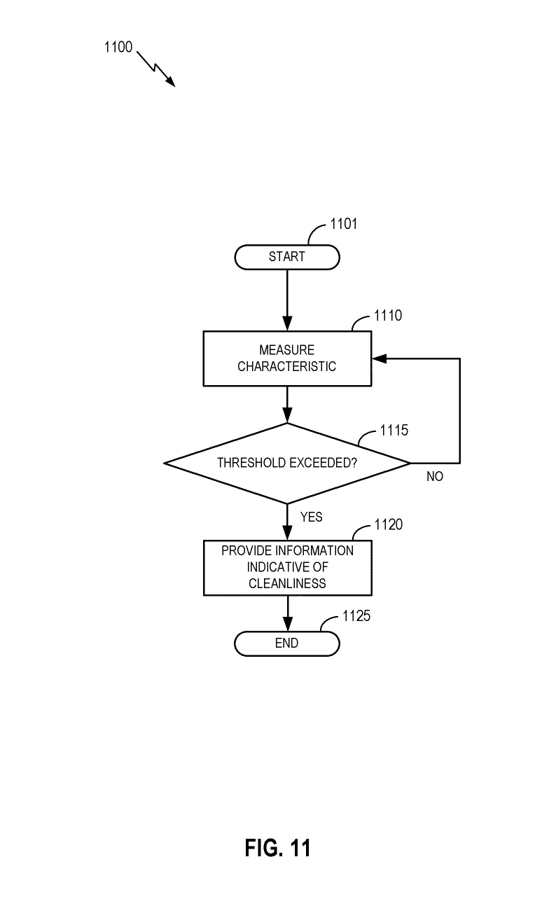

FIG. 11 depicts a method for determining cleanliness of a wearable device or an associated garment. In a method 1100 of operating a wearable device for monitoring cleanliness of the wearable device or an associated garment, a user positions the wearable device on the user's body 1101. The device then measures, based on output of an ion selective field effect transistor in the wearable device, a characteristic of a fluid present on the user's skin 1110. This characteristic is then used to determine an amount of residue buildup on the wearable device or the garment. If the amount of residue is determined to exceed a threshold amount 1115, then the device provides, via a user interface of the wearable device or an associated client device, information indicative of a cleanliness of the wearable device or the garment 1120. The device continues to monitor the characteristic until the user removes the device from the user's body 1125.

### Determining a State of Hydration

### Example C

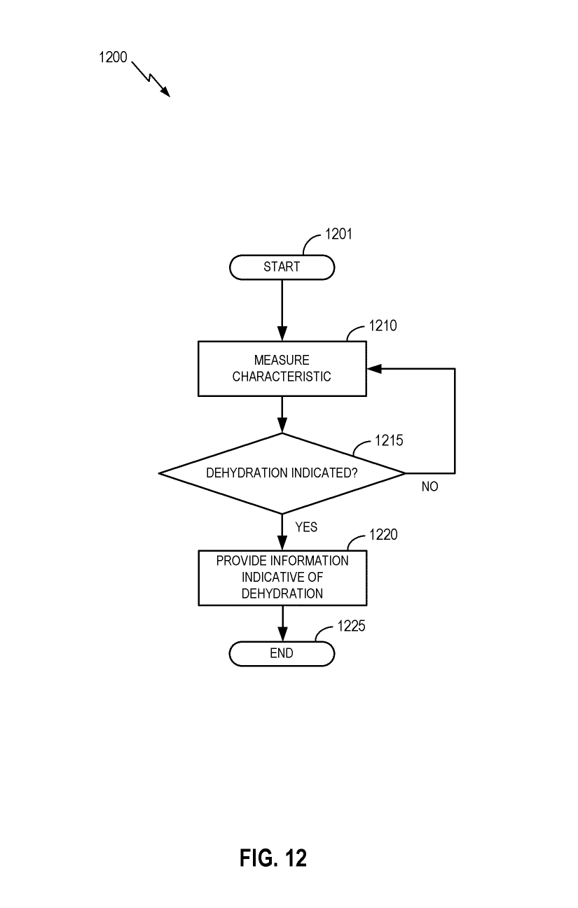

FIG. 12 depicts a method for determining a user's state of hydration. In a method 1200 of operating a wearable device for determining a user's state of hydration, a user positions the wearable device on the user's body 1201. The device then measures, based on output of an ion selective field effect transistor in the wearable device, an ion concentration of a fluid at a surface of the user's skin 1210. This measured ion concentration is then used to determine a state of hydration of the user. If the state of hydration is determined to be a dehydrated state 1215, then the device provides, via a user interface of the wearable device or an associated client device, information indicative of the dehydrated state 1220. The device continues to monitor the user's state of hydration until the user removes the device from the user's body 1225.

### Determining Skin Health

### Example D

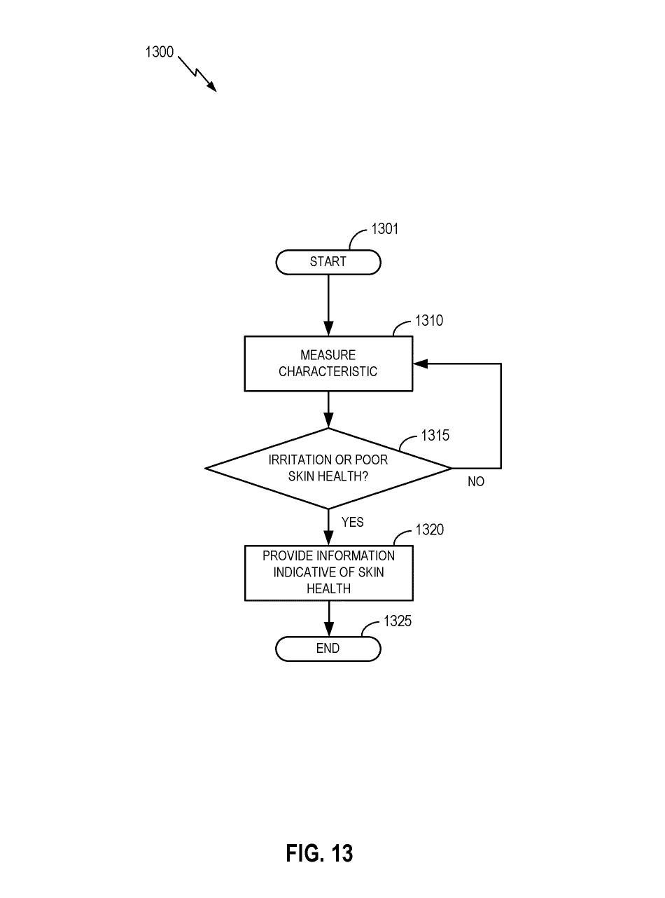

FIG. 13 depicts a method for determining skin health. In a method 1300 of operating a wearable device for determining a user's skin health, a user positions the wearable device on the user's body 1301. The device then measures, based on output of an ion selective field effect transistor of the wearable device, a characteristic of a fluid at a surface of the user's skin, wherein the characteristic is selected from the group consisting of a pH and an ion concentration 1310. This characteristic is then used to determine an indicator of health of the user's skin. If it is determined that the user's skin is irritated or in poor health 1315, then the device provides, via a user interface of the wearable device or an associated client device, information indicative of the health of the user's skin 1320. The device continues to monitor the user's state of hydration until the user removes the device from the user's body 1325.

### Example 1

### Skin pH

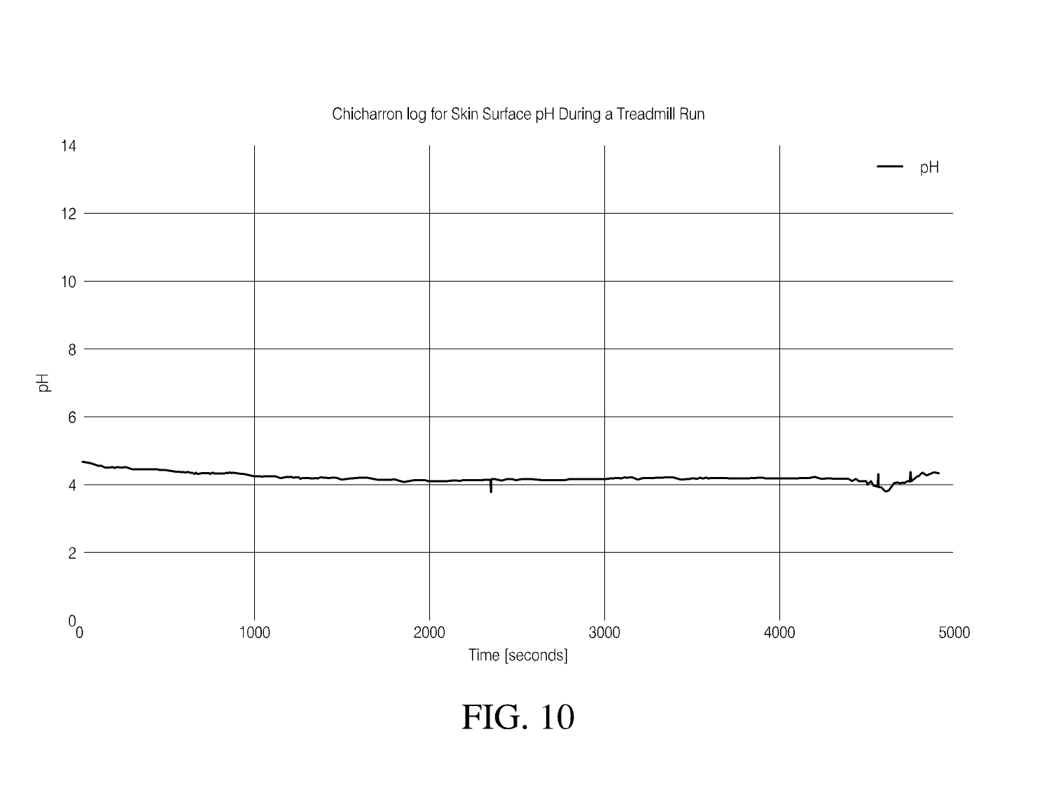

Data was collected with a prototype device incorporating a commercially available pH sensor (Horiba LAQUA™). Data was obtained from a human subject wearing the prototype device on his wrist while walking a treadmill. A plot of pH as a function of time is provided in FIG. 10. The data show that brief inflections in skin surface pH were detected, and that the sensor signal is not disrupted by vigorous motion.

### Other Considerations

While various embodiments of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. Thus the breadth and scope of the present disclosure should not be limited by any of the above-described exemplary embodiments.

It will be appreciated that, for clarity purposes, the above description has described embodiments with reference to different functional units. However, it will be apparent that any suitable distribution of functionality between different functional units may be used without detracting from the invention. For example, functionality illustrated to be performed by separate computing devices may be performed by the same computing device. Likewise, functionality illustrated to be performed by a single computing device may be distributed amongst several computing devices. Hence, references to specific functional units are only to be seen as references to suitable means for providing the described functionality, rather than indicative of a strict logical or physical structure or organization.

Embodiments of the present disclosure are described herein with reference to flowchart illustrations of methods, apparatus, and computer program products. It will be understood that each block of the flowchart illustrations, and combinations of blocks in the flowchart illustrations, can be implemented by execution of computer program instructions. These computer program instructions may be loaded onto a computer or other programmable data processing apparatus (such as a controller, microcontroller, microprocessor or the like) in a sensor electronics system to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks presented herein.

It should be appreciated that all methods and processes disclosed herein may be used in connection with the wearable device when operated in either a continuous or intermittent mode. It should further be appreciated that the implementation and/or execution of all methods and processes may be performed by any suitable devices or systems, whether local or remote. Further, any combination of devices or systems may be used to implement the present methods and processes.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least,’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities, values, amounts, and so forth used in the specification are to be understood as being modified in all instances by the term ‘about.’ Accordingly, unless indicated to the contrary, the numerical parameters set forth herein are approximations that may vary depending upon, e.g., measurement techniques or individual physiology. At the very least, and not as an attempt to limit the application of the doctrine of equivalents to the scope of any claims in any application claiming priority to the present application, each numerical parameter should be construed in light of the number of significant digits and ordinary rounding approaches.

Information and signals disclosed herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logical blocks, and algorithm steps described in connection with the embodiments disclosed herein may be implemented as electronic hardware, computer software, or combinations of both. To clearly illustrate this interchangeability of hardware and software, various illustrative components, blocks, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The techniques described herein may be implemented in hardware, software, firmware, or any combination thereof. Such techniques may be implemented in any of a variety of devices, such as, for example, wearable devices, wireless communication device handsets, or integrated circuit devices for wearable devices, wireless communication device handsets, and other devices. Any features described as devices or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

Processor(s) in communication with (e.g., operating in collaboration with) the computer-readable medium (e.g., memory or other data storage device) may execute instructions of the program code, and may include one or more processors, such as one or more digital signal processors (DSPs), general purpose microprocessors, ASICs, field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, for example, a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein. Also, the techniques could be fully implemented in one or more circuits or logic elements.

The techniques of this disclosure may be implemented in a wide variety of devices or apparatuses, including a wearable device, a wireless handset, an integrated circuit (IC) or a set of ICs (e.g., a chip set). Various components, or units are described in this disclosure to emphasize functional aspects of devices configured to perform the disclosed techniques, but do not necessarily require realization by different hardware units. Rather, as described above, various units may be combined in a hardware unit or provided by a collection of inter-operative hardware units, including one or more processors as described above, in conjunction with suitable software and/or firmware.

Although the foregoing has been described in connection with various different embodiments, features or elements from one embodiment may be combined with other embodiments without departing from the teachings of this disclosure. However, the combinations of features between the respective embodiments are not necessarily limited thereto. Various embodiments of the disclosure have been described. These and other embodiments are within the scope of the following claims.

What is claimed is:

1. A wearable device for monitoring skin health, comprising:
an ion selective field effect transistor; and
a reference electrode, wherein the ion selective field effect transistor and the reference electrode are configured to be in direct contact with a user's skin.

2. The wearable device of claim 1, wherein the reference electrode is selected from the group consisting of an Ag/AgCl electrode, an Ag/AgCl plastic composite electrode, an Ag/AgCl gel electrode, an Ag/AgCl electrode coated with a permeable membrane, a polypyrrole electrode, and a poly(3,4-ethylenedioxythiophene) electrode.

3. The wearable device of claim 1, wherein the reference electrode comprises a conductive polymer material, wherein the conductive polymer is undoped or doped.

4. The wearable device of claim 3, wherein the conductive polymer material is doped with one or more mediators.

5. The wearable device of claim 4, wherein the one or more mediators comprise ferrocene and/or ferrocene derivatives.

6. The wearable device of claim 1, further comprising a temperature sensor, wherein the temperature sensor is integrated with the ion selective field effect transistor, and wherein the temperature sensor is configured to be in direct contact with the user's skin when the wearable device is in use.

7. The wearable device of claim 1, wherein the ion selective field effect transistor is incorporated into a housing, wherein a portion of the ion selective field effect transistor is situated on a protrusion of the housing configured to enhance skin contact with the portion.

8. The wearable device of claim 1, wherein at least one of the ion selective field effect transistor and the reference electrode are configured for integration into a wristband, a sports bra, or a waistband.

9. The wearable device of claim 1, wherein the ion selective field effect transistor is configured to monitor a first characteristic of a fluid at a surface of the user's skin, wherein the monitoring is continuous and long term, and wherein the characteristic is selected from the group consisting of pH, electrolytic conductivity, Na⁺ concentration, and K⁺ concentration.

10. The wearable device of claim 9, further comprising at least one additional ion selective field effect transistor, wherein the additional ion selective field effect transistor is configured to monitor a second characteristic of fluid at a surface of the user's skin, wherein the second characteristic is different from the first characteristic.

11. A method of operating a wearable device for monitoring skin health, the wearable device comprising an ion selective field effect transistor, a reference electrode, and a user interface, the method comprising:
measuring, based on output of the ion selective field effect transistor, a characteristic of a fluid at a surface of a user's skin, wherein the characteristic is selected from the group consisting of a pH and an ion concentration;
determining, based on the measured ion concentration of the fluid, an indicator of health of the user's skin; and
providing, via the user interface, information indicative of the health of the user's skin.

12. The method of 11, wherein a pH of 6 or greater is an indicator of skin irritation or poor skin health.

13. The method of 11, wherein a specific pH threshold value is an indicator of skin irritation or poor skin health, and wherein the specific pH threshold value is user configured or user specified.

14. The method of 11, wherein a specific pH threshold value is an indicator of skin irritation or poor skin health, and wherein the specific pH threshold value is set by an algorithm and/or logic on the wearable device or a server in communication with the wearable device.

15. The method of claim 14, wherein the server is a cloud software service.

16. The method of 11, further comprising sensing a UV absorption value for the user's skin, wherein the determining comprises determining, based on output of the ion selective field effect transistor and the UV sensor, an indicator of health of the user's skin.

17. The method of 11, wherein the user interface comprises at least one of a display, a light-emitting circuit, a sound-producing circuit, and a haptic drive circuit.

18. The method of 11, wherein the wearable device further comprises a transceiver configured to communicate with a client device.

19. The method of 11, wherein the client device comprises one of a personal computer, a mobile phone, and a tablet computing device.

20. A wearable device for monitoring skin health, comprising:
an ion selective field effect transistor;
a reference electrode; and
a user interface;
at least one processor; and
a memory storing computer-executable instructions for controlling the at least one processor to:
measure, based on output of the ion selective field effect transistor, a characteristic of a fluid at a surface of a user's skin, wherein the characteristic is selected from the group consisting of a pH and an ion concentration;
determine, based on the measured characteristic of the fluid, an indicator of health of the user's skin; and
provide, via the user interface, information indicative of the health of the user's skin.

21. The wearable device of claim 20, wherein the processor is configured to sample the output of the ion selective field effect transistor at a faster rate when the user is physically active than when the user is sedentary.

22. The wearable device of claim 20, wherein the processor is configured to sample the output at the faster rate after the user has been physically active for at least a predetermined period.

23. The wearable device of claim 20, wherein the predetermined period is ten or more minutes.

24. The wearable device of claim 23, further comprising a temperature sensor, wherein the temperature sensor is integrated with the ion selective field effect transistor, and wherein the temperature sensor is configured to be in direct contact with the user's skin when the wearable device is in use.

25. The wearable device of claim 20, wherein the reference electrode is incorporated into a housing.

26. The wearable device of claim 20, wherein the ion selective field effect transistor is incorporated into a housing, wherein a portion of the ion selective field effect transistor is situated on a protrusion of the housing configured to enhance skin contact with the portion.

27. The wearable device of claim 20, wherein at least one of the ion selective field effect transistor and the reference electrode are configured for integration into a wristband, a sports bra, or a waistband.

28. The wearable device of claim 27, further comprising at least one additional ion selective field effect transistor, wherein the additional ion selective field effect transistor is configured to monitor a second characteristic of fluid at a surface of the user's skin, wherein the second characteristic is different from the first characteristic.

29. A wearable device for monitoring a user's skin health, comprising:
an ion selective field effect transistor;
a reference electrode; and
a user interface;
at least one processor;
one or more biometric sensors configured to determine a physiological metric of the user, wherein the measured physiological metric is used by the processor to improve an accuracy of the information provided via the user interface.

30. The wearable device of claim 29, wherein the processor is configured to sample the output of the ion selective field effect transistor at a faster rate when the user is physically active than when the user is sedentary.
